(19) United States
(12) Patent Application Publication
(10) Pub. No.: US 2008/0254002 A1
(43) Pub. Date: Oct. 16, 2008

(54) Bone Marrow Derived Oct3/4+ Stem Cells

(76) Inventors: Jay M. Edelberg, New York, NY (US); Benedetta A. Pallante, New York, NY (US)

Correspondence Address: Hoffmann&Baron, 6900 Jricho Turnpike, Syosset, NY, 11791, US

(21) Appl. No.: 11/661,773

(22) PCT Filed: Sep. 2, 2005
(86) PCT No.: PCT/US05/31547

§ 371 Date: Jan. 28, 2008

Related U.S. Application Data
(60) Provisional application No. 60/607,127, filed on Sep. 3, 2004.

(51) Int. Cl. A61K 35/12; C12N 5/06; A61P 9/00

(52) U.S. Cl. 424/93.7; 435/325; 435/384

(57) ABSTRACT

The invention provides bone-marrow derived stem cells, e.g., cardiomyocyte precursor cells, differentiated cardiomyocytes generated from the precursor cells, and a method for treating cardiac dysfunction in a subject by administering such cells.

### GOVERNMENT FUNDING

The invention described in this application was made with funds from the National Institute of Health, Grant Numbers AG19738 and AG20918. The United States government has certain rights in the invention.

### FIELD OF THE INVENTION

The invention relates to bone marrow-derived stem cells, cardiomyocyte precursor cells and cardiomyocytes generated from the bone-marrow derived cells. Such cells can be genetically modified to express useful gene products. The invention further relates to methods for using these cells for treating cardiac diseases and other conditions.

### BACKGROUND OF THE INVENTION

Pluripotent stem cells are a valuable resource for research, drug discovery and therapeutic treatments, including transplantation. These cells and their mature progeny can be used to study signaling events that regulate differentiation processes, identify and test drugs for lineage-specific beneficial or cytotoxic effects, or replace tissues damaged by disease or an environmental impact. The current state of pluripotent stem cell biology and the medicinal outlook, however, are not without drawbacks or free from controversy.

The use of pluripotent stem cells from fetuses, umbilical cords or embryonic tissues derived from in vitro fertilized eggs raises ethical and legal questions in the case of human materials, poses a risk of transmitting infections and/or may be ineffective because of immune rejection. One way to circumvent these problems is by exploiting autologous stem cells. In this context, it has been reported that bone marrow contains cells that appear to have the ability to trans-differentiate into mature cells belonging to cell lineages other than those of the blood. However, recent studies have questioned the existence of such a trans-differentiation and raised the possibility that the emerging mature cells result from fusion of stem cells with resident tissue cells.

Recent progress in the area of stem cell research implies that hematopoietic stem cells (HSC) might potentially be used in regenerative medicine. HSCs can be isolated from adult mammals, including humans. They reside in the bone marrow and under some conditions migrate to other tissues through the blood. HSCs are also normally found in the fetal liver and spleen and in umbilical cord and placenta blood. There is a growing body of evidence that HSCs are plastic, i.e., are able to participate in the generation of tissues other than those of the blood system. For example, studies have demonstrated the potential of bone marrow-derived cells to give rise to new heart muscle cells improving postinfarction cardiac function in clinical studies (Assumus et al., 2002; Strauer et al., 2002). However, these results can be attributed, in part, to the fusion of the bone marrow-derived cells with preexisting cells in intact myocardium (Alvarez-Dolado et al., 2003; Terada et al., 2002) or direct conversion from one differentiated cell type to another (transdifferentiation) (Eisenberg et al., 2003). Moreover, the stem cell compartment in human bone marrow is highly complex, comprising both CD34⁺ and CD34⁻ HSCs, mesenchymal progenitors, and perhaps other cell types whose activities remain to be defined.

Thus, needs exist in the art to isolate, culture, sustain, propagate, and differentiate adult stem cells in order to develop cell types suitable for a variety of uses. Such uses may include the use of autologous stem cells for the treatment of diseases and amelioration of symptoms of diseases, for example, cardiac diseases.

### SUMMARY OF THE INVENTION

The present inventors have discovered a bone marrow subpopulation of cells expressing Oct3/4, a marker associated with an undifferentiated/pluripotential state. For example, the bone-marrow derived stem cells of the invention can spontaneously form embyroid bodies that give rise to cardiac myocytes independent of the need for pre-existing cardiac myocytes. Temporally, the expression of phenotypic stem cell genes (Oct3/4 and Dppa3 and Dppa4) was inversely correlated with the induction of cardiac myocyte specific genes (β and then α myosin heavy chains), demonstrating that the adult bone marrow contains cells expressing markers associated with extended nuclear plasticity and capable of functioning as cardiac stem cells. As used herein, the phrase “embryoid bodies” or “EBs” refers to collections of cells formed from the aggregation or clustering of cultured bone-marrow derived Oct3/4⁺ stem cells as described herein. EBs have a three dimensional morphology, e.g., they can be a solid or a cystic embryoid body.

As discussed herein, the bone marrow-derived cardiac myocytes arise from Oct3/4 cells that have formed embryoid body-like aggregates. These findings point to the use of the Oct3/4 cells alone or in these aggregates as the cardiac myocyte stem cells for cardiac tissue repair/regeneration. Moreover, the similarity of the Oct3/4 cells with embryonic stem cells suggests that these cells and/or the aggregates may be used for applications in which embryonic stem cells are presently employed. Such applications include the generation of endoderm, mesoderm and ectoderm cells, tissue repair and tissue regeneration. In addition, as embryonic stem cells are used for the derivation of embryos and germ cell generation, the Oct3/4 cells may be able to give rise to germ cells as well.

Accordingly, the present invention provides an isolated mammalian bone-marrow derived stem cell that expresses Oct3/4. In one embodiment, the stem cell also expresses Dppa 3/Stella, Dppa4, or both. In another embodiment, the stem cell expresses at least one of Dppa3/Stella, Dppa4, Dppa5 or a combination thereof. In addition, the stem cell may further express at least one receptor selected from the group consisting of a receptor for vascular endothelial growth factor (VEGF) or fibroblast growth factor (FGF), for example, VEGF receptor (VEGFR)-2 (also known as fetal liver kinase-1, Flk-1), and/or fibroblast growth factor-1 (FGFR-1) and/or c-Kit. In another embodiment, the stem cell of the invention does not express CD34, Sca1 or a combination thereof. Such a stem cell may in addition have alkaline phosphatase activity. For example, the invention provides a bone-marrow derived stem cell that is Sca1⁻/CD34⁻/c-Kit⁺/Flk1⁺/FGFR⁺. In other embodiments, the bone marrow cells of the invention that give rise to cardiac myocytes are Oct3/4⁺ Sca1⁻ CD34⁻ c-Kit^{+/-} Flk1^{+/-} FGFR^{+/-}.

In addition, the present invention provides an isolated mammalian bone-marrow derived cardiomyocyte that expresses β myosin heavy chain, α myosin heavy chain, cardiac troponin T, or a combination thereof, wherein the cardiomyocyte was generated from a mammalian bone-marrow derived stem cell that expresses Oct3/4. Such a cardiomyocyte may further express Oct3/4, Dppa 3/Stella, Dppa 4, β myosin heavy chain, α myosin heavy chain, or a combination thereof. In addition, a cardiomyocyte of the invention has spontaneous beating and/or chronotropic activity. In some embodiments, the bone marrow cells of the invention that give rise to cardiac myocytes are Oct3/4⁺ Sca1⁻ CD34⁻ c-Kit^{+/-} Flk1^{+/-} FGFR^{+/-}.

The present invention further provides a bone-marrow derived embryoid body comprising a cardiomyocyte of the invention, as described herein. Further provided is a bone-marrow derived embryoid body that includes a stem cell of the invention. In one embodiment, the bone-marrow derived embryoid body has at least one cell that expresses Oct3/4, SSEA1, alpha-fetoprotein, or a combination thereof. In another embodiment, the bone-marrow derived embryoid body comprises at least one cell that is Sca1⁻/CD34⁻/c-Kit⁺/Flk1⁺/FGFR⁺, and preferably at least one cell that is Oct3/4⁺ Sca1⁻/CD34⁻/c-Kit⁺/Flk1⁺/FGFR⁺. A bone-marrow derived embryoid body of the invention may also have alkaline phosphatase activity.

Further is provided a composition comprising mammalian cardiomyocytes derived from Oct3/4⁺ bone-marrow cells, wherein the cardiomyocytes express at least one marker selected from the group Oct3/4⁺, Dppa 3/Stella, Dppa 4, FGFR-1, VEGFR, flk1, c-Kit, alpha fetoprotein (AFP), β myosin heavy chain and α myosin heavy chain. In one embodiment, the composition comprises mammalian cardiomyocytes derived from Oct3/4⁺ bone-marrow cells that do not express CD34 or Sca1. In other embodiments, at least one of cardiac myocytes is Oct3/4⁺ Sca1⁻CD34⁻ c-Kit^{+/-}Flk1^{+/-}FGFR^{+/-}.

The invention also provides a method for treating cardiac dysfunction in a patient having or at risk for developing cardiac dysfunction comprising administering to the mammal a therapeutically effective amount of bone-marrow derived stem cells or differentiated cardiomyocytes derived from the cells, wherein the cells express at least one marker selected from the group Oct3/4, Dppa 3/Stella, Dppa 4, FGFR-1, VEGFR, flk1, c-Kit, alpha fetoprotein (AFP), β myosin heavy chain and α myosin heavy chain. In some embodiments, the cells employed express Oct3/4. In other embodiments, the stem cells or differentiated cardiomyocytes derived from those cells are Oct3/4⁺ Sca1⁻CD34⁻ c-Kit^{+/-}Flk1^{+/-}FGFR^{+/-}.

The cardiovascular dysfunction can be any cardiac or vascular disease or condition. For example, the cardiovascular dysfunction can be myocardial infarction, ischemia, peripheral vasculature disorder (PVD), stroke, atherosclerosis, arrhythmia, heart failure, tachycardia, or congestive heart failure.

In addition, the invention provides a method of making a cardiomyocyte comprising obtaining stem cells that express Oct3/4 from mammalian bone-marrow and culturing the cells in a medium comprising an appropriate amount of at least one growth factor under appropriate conditions for a sufficient period of time to promote differentiation of the stem cell into a cardiomyocyte. For example, the growth factor is vascular endothelial growth factor (VEGF), fibroblast growth factor-2 (FGF-2), or a combination thereof.

The invention also provides a method for treating a cardiac dysfunction in a mammal comprising providing Oct3/4⁺ cardiomyocyte precursor cells obtained from bone marrow stem cells collected from the mammal and administering the cardiomyocyte precursor cells to the mammal. One embodiment of the method involves culturing the Oct3/4⁺ cardiomyocyte precursor cells in a culture medium comprising VEGF and FGF-2 under conditions that induce the cells to differentiate into cardiomyogenic cells, monitoring the differentiation state of the cardiomyogenic cells and administering the cardiomyogenic cells to the mammal.

Additionally is provided a method for making a bone-marrow derived embryoid body comprising obtaining cardiomyocyte precursor cells from mammalian bone marrow, wherein the cells express Oct3/4 and culturing the cells in a medium comprising an appropriate amount of at least one growth factor under appropriate conditions for a sufficient period of time to provide an embryoid body. For example, the bone-marrow derived embryoid body comprises at least one cell that expresses Oct3/4. In another example, the bone-marrow derived embryoid body further comprises at least one cell that expresses at least one of Dppa 3/Stella or Dppa4. The bone-marrow derived embryoid body of the invention may further comprise at least one cell that expresses at least one receptor selected from the group consisting of a receptor for vascular endothelial growth factor (VEGF) or fibroblast growth factor (FGF), for example, fetal liver kinase 1 (Flk-1), and/or fibroblast growth factor receptor-1 (FGFR-1), as well as c-Kit. In one embodiment, the bone-marrow derived embryoid body of the invention comprises at least one cell having alkaline phosphatase activity. In yet another embodiment, the bone-marrow derived embryoid body comprises cells that are Sca1⁻/CD34⁻ c-Kit⁺/Flk1⁺/FGFR⁺. In other embodiments, the bone marrow derived embryoid body comprises cells that are Oct3/4⁺Sca1⁻CD34⁻ c-Kit^{+/-}Flk1^{+/-}FGFR^{+/-}.

A method of making an embryoid body is provided, which method comprises obtaining Oct3/4⁺ cells from mammalian bone-marrow and culturing the cells under conditions suitable to produce an embryoid body that comprises cells where at least some of the cells are Sca1⁻/CD34⁻/c-Kit⁺/Flk1⁺/FGFR⁺. In some embodiments, the embryoid body produced by the method comprises cells where at least some of the cells are Oct3/4⁺Sca1⁻CD34⁻ c-Kit^{+/-}Flk1^{+/-}FGFR^{+/-}.

The invention further provides a germ cell generated in vitro from bone-marrow derived Oct3/4⁺ stem cell, for example, a spermatocyte or oocyte that is Sca1⁻/CD34⁻/c-Kit⁺/Flk1⁺/FGFR⁺. In some embodiments, the spermatocytes or oocyte is Oct3/4⁺ Sca1⁻CD34⁻ c-Kit^{+/-}Flk1^{+/-}FGFR^{+/-}.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIG. 1A-D shows that bone marrow cells engraft into heart tissue and concentrate around vascular structures. Bone marrow cells of B6.129Sv-Gtrosa26 (Rosa-26) mice were co-cultured with day 14 C57BL/6 fetal hearts in the presence of vascular endothelial growth factor (VEGF) and fibroblast growth factor (FGF)-2 for 2 days (FIG. 1A) and 7 days (FIG. 1B). As shown by X-Gal staining, engraftment of cells was maximal at day 7 and was greatest in vascular structures (FIG. 1B). Engraftment into cardiac tissue was also confirmed by co-culturing C57BL/6 bone marrow cells, pre-labeled with the fluorescent dye 5-chloromethyl fluorescein diacetate (CMFDA), with wild type fetal hearts, for 7 days (FIG. 1C-D). Scale bar, 100 μm.

FIG. 2A-F depicts the in vitro differentiation of bone marrow cells into cardiomyocytes and Embryoid Body-like structures (EBs). Mesenchymal stromal cells (FIG. 2B) and haematopoietic stem cells (FIG. 2C), derived from mouse bone marrow, were co-cultured in the presence of vascular endothelial growth factor (VEGF) and FGF-2 for 2 days (FIGS. 2A and 2D), 7 days (FIGS. 2B and 2E) and 14 days (FIGS. 2C and 2F). Under these conditions, spheroidal bodies (FIG. 2D) differentiated into contracting cardiomyocytes (FIG. 2E; black arrow) and EBs (FIGS. 2E and 2F; white arrows). Scale bar, 100 μm.

FIG. 3A-C depicts immunostaining for alpha fetal protein (AFP, FIG. 3A) with nuclear counterstain (DAPI; 4′,6-diamidono-2-phenylindule, dilactate, FIG. 3B) at day 7 and day 14 that confirmed the development of phenotypic embryoid-like bodies in bone marrow cell cultures. FIG. 3C provides a merged image showing both AFP and DAPI staining.

FIG. 4A-F shows that bone marrow cells positively stained for the pluripotency marker Oct3/4. Epifluorescent images showed positive cytoplasmic staining for Oct3/4 in freshly isolated cells (FIG. 4D) and ES-like clusters (FIG. 4E). Cytoplasmic staining pattern for Oct3/4 was similar to that observed in oocytes (FIG. 4F). Cells were prepared on cytospin slides or were grown in wells. Specimens were fixed with methanol/acetone (1:1), stained with anti-human recombinant Oct3/4 and fluoroscein isothiocyanate (FITC) (FIG. 4D-E) or Texas red (TXR) (FIG. 4F) conjugated secondary antibody. In control samples, incubation with primary antibody was omitted (FIG. 4A-C, inserts). All cells were counterstained with DAPI (FIG. 4A-C).

FIG. 4G illustrates the expression of OCT3/4 and Dppa3-5 molecular markers for pluripotency after culturing total bone marrow for 1 day, 5 days, 14 days and 21 days. These results were also confirmed by reverse-transcription polymerase chain reaction (RT-PCR). Expression was modulated during culture at different time points for the different genes.

FIG. 5A-E depicts bone marrow cells that have the potential to differentiate spontaneously into cell types from the three germ layers. Cells were cultured in chamber slides for 14 days with FGF-2 and VEGF. Indirect immunolabelling confirmed presence of cells of cardiomyocytes (troponin-T/C⁺, FIG. 5A), smooth muscle cells (SM-alpha actin⁺, FIG. 5B), endothelial cells (Pecam-1⁺, FIG. 5C), all of mesodermal origin. The presence of cells of ectodermal (neurofilament⁺, FIG. 5D) and endodermal origin (AFP⁺, FIG. 5E) was also confirmed.

FIG. 6A-F shows that cardiac myocytes originate from bone marrow cell 3D aggregates (FIGS. 6A and 6D). Bone marrow 3D cell aggregates with cardiomyogenic potential form on patches of developing stroma cells (FIGS. 6B and 6E). By day 7, clusters of cells with chronotropic activity start appearing at the periphery of ⅓ of bone marrow aggregates (FIGS. 6C and 6F). At day 10, clusters of cells with chronotropic activity became larger, started detaching, and are observed in suspension until day 14 of culture when beating activity ceased (FIGS. 6C and 6F). The inserts in FIGS. 6C and 6F show cardiac myocytes, identified by positive immunostaining for the cardiac marker troponin-T, found at the periphery of bone marrow cell aggregates and in detaching cell clusters. For each image shown in FIG. 6, bone marrow aggregates are indicated by white arrows; cardiac myocyte clusters by black arrows; bar 50 μm.

FIG. 7A-H illustrates that cardiac myocytes originate from Oct-3/4⁺ bone marrow (BM)-derived embryoid bodies (EBs). Cell-aggregates form from total BM-cell cultures (day-5, FIGS. 7A, 7E) and generate (day-7, FIGS. 7B, 7F) clusters of troponin-T⁺ cardiac myocytes that detach and are observed in suspension (day-10, FIGS. 7C, 7G inserts). Purified day-5 BM-derived aggregates (FIG. 7D) retain ability to generate troponin-T⁺ cardiac myocytes. BM-derived aggregates (FIG. 7H). The arrows identify BM-derived cell aggregates (embryoid-like bodies), and the arrowheads identify cardiomyocyte clusters.

FIG. 8A-B shows that BM-derived cell aggregates (FIG. 8A) are similar to embryonic stem cell-derived embryoid bodies (EBs, FIG. 8B).

FIG. 9A-B shows that BM-derived cell aggregates express the ES-cell marker Oct-3/4. FIG. 9A provides an image of a gel with electrophoretically separated products of transcripts amplified by reverse transcriptase-polymerase chain reaction (RT-PCR). As shown in the first (d7) and second (d14) lanes, Oct-3/4 is expressed in bone marrow cells 7 days after isolation, but reduced levels of Oct-3/4 expression were observed by fourteen days. Oct-3/4 expression in embryonic stem (ES) cells and ovarian tissues is shown as a positive control. Liver expression of Oct-3/4 is shown as a negative-control. FIG. 9B graphically illustrates quantitative Oct-3/4 expression observed by quantitative RT-PCR. The Arbitrary Units (AU) of Oct-3/4 expression employed were calculated as the quantity of RT-PCR product relative to day-7 BM aggregates.

FIG. 10A-H illustrates that BM-derived aggregates express Oct-3/4. FIG. 10A provides an image of an oocyte as a positive-control for Oct-3/4 expression. No Oct-3/4 expression was observed in cumulus cells that are shown in the inserts (a negative-control). FIG. 10B shows Oct-3/4 expression in a 7 day bone marrow derived embryoid-like body. FIG. 10C shows alkaline phosphatase (AP) expression in a 7 day bone marrow derived embryoid-like body. FIG. 10D shows a merged image illustrating Oct-3/4 and troponin T expression in a 7 day bone marrow derived embryoid-like body. FIG. 10E provides an image of embryonic stem cells in an embryoid body as a positive-control for Oct-3/4 expression. FIG. 10F shows Oct-3/4 expression in a 14 day bone marrow derived embryoid-like body. FIG. 10G shows alpha-fetoprotein (AFP) expression in a 14 day bone marrow derived embryoid-like body. FIG. 10H shows a merged image illustrating Oct-3/4 and troponin T expression in a 14 day bone marrow derived embryoid-like body. As shown in FIGS. 10D and 10H, cardiac troponin-T colocalizes with Oct-3/4 in BM-derived EBs. Merged images show DAPI nuclear stain (blue in original). In the original, Oct-3/4 expression was detected by green staining and Troponin-T⁺ cells were detected as red staining. The arrows identify troponin T⁺ cells and the arrowheads identify troponin-T⁺/Oct-3/4⁺ cells. Bars, 50 μm

FIG. 11A-D illustrates that fresh isolates of mouse bone marrow cells contain cells that express the pluripotency marker Oct-3/4, though Oct-3/4 expression is rarer (0.05±0.03%) and it is expressed at lower levels than in ES-cells, as detected by in situ immunostaining of embryonic stem cells and bone marrow cells. FIGS. 11A and 11B show DAPI staining of DNA (blue in the original) in embryonic stem cells (FIG. 11A) and freshly isolated bone marrow cells (FIG. 11B). FIGS. 11C and 11D show the same fields of embryonic stem cells (FIG. 11C) and bone marrow cells (FIG. 11D) immunostained for Oct-3/4-FITC (green). Bar, 50 um.

FIG. 12A shows that bone marrow (BM) cell expression of Oct-3/4 is down-regulated during cardiomyogenesis in vitro. An image of a gel with electrophoretically separated RT-PCR products shows that Oct-3/4 and the other pluripotency-associated genes, Dppa3 and Dppa4, are expressed at the time of bone marrow cell isolation and become down-regulated within several days to several weeks in culture. Ovarian and liver expression of Oct-3/4 is shown as positive and negative RT-PCR controls, respectively.

FIG. 12B is an image of gel with electrophoretically separated RT-PCR products from bone marrow cells cultured over time shows that Oct-3/4 levels inversely correlate with the induction of PDGF-B and of the cardiac-specific markers for adult α-myosin heavy chain (α-MHC), and fetal β-myosin heavy chain (β-MHC).

FIG. 12C graphically illustrates Oct-3/4, β-MHC, α-MHC and PDGF-B expression levels in bone marrow cells as a function of the number of days in culture. The Arbitrary Units (AU) employed for expression levels were calculated from quantitative RT-PCR levels observed relative to the quantitative RT-PCR levels observed for Day-0 (Oct-3/4 only) or Day-14 bone marrow cell cultures.

FIG. 13A-D illustrates that Oct-3/4⁺ bone marrow (BM) cells also express c-Kit, but not significant levels of CD34 or Sca-1. FIG. 13A shows Oct-3/4+ BM cells (FITC, green in original) co-express c-Kit (red in original). FIG. 13B shows Oct-3/4+ BM cells do not express significant amounts of CD34. FIG. 13C shows Oct-3/4+ BM cells do not express significant amounts of Sca1. FIG. 13D graphically illustrates the percentage of cells expression Oct-3/4⁺, cKit and Sca1 in bone marrow isolates.

FIG. 14A-E illustrates that Oct-3/4⁺ bone marrow cells also express Flk-1 and FGFR-1, but not significant levels of PDGFRα (Oct-3/4⁺ expression was green while Flk-1, FGFR-1 and PDGFRα expression was red in the original). Arrowheads identify Flk-1 and FGFR-1 expression in Oct-3/4⁺ bone marrow cells. FIG. 14A shows Flk1 expression in Oct-3/4⁺ bone marrow cells (arrowhead). FIG. 14B shows FGFR1 expression in Oct-3/4⁺ bone marrow cells (arrowhead). FIG. 14C shows a merged image illustrating Flk1 and FGGR1 expression in an Oct-3/4⁺ bone marrow cell (arrowhead). FIG. 14D shows no significant levels of PDGFRα expression in Oct-3/4⁺ bone marrow cells (arrow). FIG. 14E graphically illustrates the percentage of cells expressing Oct-3/4, Flk-1 and FGFR-1 in bone marrow isolates.

FIG. 15A-D illustrates Day-13 bone marrow cell-aggregate expression of c-Kit (FITC, green, FIG. 15A arrowheads), Flk-1 (FITC, green, FIG. 15B-C) and FGFR-1 (Texas Red, arrowheads FIG. 15B-C). Flk-1+ cells also surrounded contracting cardiomyocytes (FIG. 15C, arrows). PDGFRα⁺ cells (FIG. 15D, arrowheads) were adjacent to the cell aggregates. All merged images also show the DNA dye DAPI (blue). Bar, 20 μm.

FIG. 16A-B illustrates that bone marrow (BM)-derived Oct-3/4⁺ cells regenerate cardiac myocytes in the heart after the heart has suffered an infarction. Donor BM cells (CMFDA, green) were injected into infarcted rat hearts with (+GF, FIG. 16B) or without (−GF, control, FIG. 16A) growth factor treatment. After four days, heart sections were obtained and stained for Oct-3/4 (Texas red) and troponin-T (Cy5, blue). High numbers of donor BM-cells survived and were observed as green-stained cells in the heart sections (FIG. 16B). Oct-3/4⁺ cells represented about one-third of donor BM cells (FIG. 16B, arrows) and contributed to about one-fourth of BM-derived troponin-T⁺ cardiac myocytes (FIG. 16B, arrowheads). Resident Oct-3/4 cells (CMFDA-negative) were also observed (FIG. 16B, circle) and occasionally gave rise to cardiomyocytes (FIG. 16B, square). Bar, 50 um.

FIG. 16C-D graphically illustrates the number/field (FIG. 16C) and the percentage (FIG. 16D) of donor bone marrow cells observed in rat heart sections obtained from rats that had received bone marrow cells cultured in growth factors (+GF, shaded bars) or bone marrow cells not cultured in growth factors (−GF, open bars). As shown in FIG. 16C, greater numbers of donor bone marrow cells are detected in the hearts of rats when those donor bone marrow cells are previously cultured in growth factors. FIG. 16D graphically illustrates the percentage of donor bone marrow cells detected in rat heart sections that express CMFDA (marker for donor bone marrow cells), Oct-3/4, and/or troponin-T. Note that essentially only the donor cells cultured in growth factors (shaded bars) express Oct 3/4 and/or troponin-T. Donor bone marrow cells cultured without growth factors (open bars) exhibit little if any expression of Oct-3/4 and troponin-T.

### DETAILED DESCRIPTION OF THE INVENTION

The present invention provides pharmaceutical compositions comprising an effective amount of bone-marrow derived cardiomyocyte precursor cells, for example, stem cells, and/or cardiomyocytes. The invention is also directed to methods for treating a cardiac disease or condition in a mammal that include administering an effective amount of the bone-marrow derived cardiomyocyte precursor cells and/or cardiomyocytes, for example, cardiomyocyte precursor cells that express Oct3/4. Moreover, the cells can be genetically engineered to express useful gene products that can further enhance restoration and health of cardiac tissues.

The present invention discloses the plasticity and potential of total bone marrow cells (TBM) in vitro and in vivo. According to the present invention, bone marrow cells are incorporated/recruited to sites of injury and generate cardiac myocytes and cardiac endothelial cells using rodent models of myocardial infarction. Indeed, the bone marrow-derived cells disclosed herein reconstitute vital critical subpopulations of endothelial progenitor cells to promote cardiac vascular function, suggesting that the cells of the bone marrow have the potential to migrate to an array of organs to generate vascular and organ-specific cell types.

The term “cardiomyocyte” is used interchangeably herein with “cardiac myocyte” and refers to any cell in the cardiac myocyte lineage that shows at least one phenotypic characteristic of a cardiac muscle cell. Such phenotypic characteristics can include expression of cardiac proteins, such as cardiac sarcomeric or myofibrillar proteins or atrial natriuretic factor, or electrophysiological characteristics. Cardiac sarcomeric or myofibrillar proteins include, for example, atrial myosin heavy chain, cardiac-specific ventribular myosin heavy chain, desmin, N-cadherin, sarcomeric actin, cardiac troponin I, myosin heavy chain, and Na-K-ATPase. Electrophysiological characteristic of a cardiomyocyte include, for example, transient K⁺ channel currents, and acetylcholine and cholera toxin responses. For example, a cardiomyocyte may spontaneously beat or may exhibit calcium transients (flux in intracellular calcium concentrations measurable by calcium imaging). A cardiomyocyte of the invention may express at least one cardiomyocyte specific marker, such as myosin heavy chain or α-myosin heavy chain. In another embodiment, a cardiomyocyte of the invention expresses at least one of the following markers: cardiac transcription factor-4 (GATA-4), cardiogenic homeodomain factor Nkx 2.5, atrial myosin light chain type 2 (MLC-2A), ventricular myosin light chain type 2 (MLC-2V), human atrial natriuretic peptide (hANP), cardiac troponin T (cTnT), cardiac troponin I (cTnI), alpha-actinin, sarcomeric myosin heavy chain (MHC), N-cadherin, beta1-adrenoceptor (beta1-AR), the myocyte enhancer factor-2 (MEF-2) family of transcription factors, creatine kinase MB (CK-MB), or myoglobin.

The cardiomyocyte derived by the present method may contain contractile elements and may be capable of “beating,” i.e., spontaneous contraction and/or beating. The cardiomyocyte of the invention is capable of electronic coupling with spontaneously contracting cardiac muscle and integrating into the syncytium of the cardiac muscle, thereby becoming a contracting cardiac muscle cell. Contraction can be induced by methods known to the art such as by changing ion concentration (e.g., by elevated K⁺), by mechanical stimulation, or by electrical stimulation.

### General Techniques

For further elaboration of general techniques useful in the practice of this invention, the practitioner can refer to standard textbooks and reviews in cell biology, tissue culture, embryology, and cardiophysiology.

With respect to tissue culture and stem cells, the reader may wish to refer to Guide to Techniques in Mouse Development (Wasserman et al. eds., Academic Press 1993); Embryonic Stem Cell Differentiation in Vitro (Wiles, Meth. Enzymol. 225: 900, 1993); Properties and uses of Embryonic Stem Cells: Prospects for Application to Human Biology and Gene Therapy (Rathjen et al., Reprod. Fertil. Dev., 10: 31 (1998)). With respect to the culture of heart cells, standard references include The Heart Cell in Culture (Pinson ed., CRC Press (1987)), Isolated Adult Cardiomyocytes (Vols. I & II, Piper & Isenberg eds., CRC Press (1989)), and Heart Development (Harvey & Rosenthal, Academic Press (1998)).

General methods in molecular and cellular biochemistry can be found in such standard textbooks as Molecular Cloning: A Laboratory Manual, 3^{rd} ed. (Sambrook et al., Cold Spring Harbor Press (2001)), Short Protocols in Molecular Biology, 4^{th} ed. (Ausubel et al., eds., John Wiley & Sons (1999)), Protein Methods (Bollag et al, I John Wiley & Sons (1996)), Nonviral Vectors for Gene Therapy (Wagner et al., eds., Academic Press (1999)), Viral Vectors (Kaplitt & Loewy, eds., Academic Press (1995)), Immunology Methods Manual (I. Lekovits, ed., Academic Press (1997)), and Cell and Tissue Culture: Laboratory Procedures in Biotechnology (Doyle & Griffiths, John Wiley & Sons (1998)). Reagents, cloning vectors and kits for genetic manipulation referred to in this disclosure are available from commercial vendors such as BioRad, Stratagene, Invitrogen, Sigma-Aldrich and ClonTech.

### The Stem Cells and Cardiomyocyte Precursor Cells of the Invention

As discussed herein, the bone-marrow derived stem cells and cardiomyocyte precursor cells of the invention are pluripotent cells that can, for example, differentiate into functional cardiomyocytes. According to the invention, the precursor cells can give rise to heart muscle cells and improve, for example, post-infarction cardiac function. As used herein, the term “cardiomyocyte precursor cells” refers to a cell that is capable (without dedifferentiation or reprogramming) of giving rise to progeny that include cardiomyocytes, and that expresses Oct3/4. The cardiomyocyte precursor cells of the invention are obtained from mammalian bone-marrow cells. Such precursor cells are capable of populating the intact, senescent bone marrow, homing to sites of cardiac angiogenic induction, restoring pathways required for vascular function, homing to sites of internal injury and facilitating re-endothelialization. These cells can restore and stimulate cardiac angiogenesis in an aging host, for example, by healing injured vascular tissues, generating cardiac myocytes and promoting the development of new cardiac endothelial tissues. The cardiomyogenic cells employed in the invention can be stem cells, cardiomyocyte precursor cells or partially differentiated cardiomyocyte precursor cells.

The cardiomyocyte precursor cells of the invention can be derived from non-embryonic bone marrow-derived cells, e.g., adult bone-marrow derived cells. By “non-embryonic” is meant fetal or postnatal. The embryonic period is considered to be early prenatal development, and specifically, in the human, the first eight weeks following fertilization. One skilled in the art would recognize that the equivalent period in other mammalian species would constitute the embryonic period.

Pluripotent stem cells are capable of developing into more than two types of mature cells, such as cardiac myocyte cells, hematopoietic cells, and at least one other type of cell. Bipotent stem cells are capable of developing into two types of mature cells, such as cardiac myocyte cells and hematopoietic cells. Progenitor cells are capable of developing into one type of mature cells, such as cardiac myocyte cells or hematopoietic cells. Pluripotent stem cells, bipotent stem cells, and progenitor cells are capable of developing into mature cells either directly or indirectly through one or more intermediate stem or progenitor cell. A “cardiomyocyte stem cell” or “cardiomyocyte precursor cell” is a stem cell that is capable of maturing into at least one type of mature cardiomyocyte cell. The cardiomyocyte stem cell may be pluripotent, bipotent, or monopotent. Monopotent cardiomyocyte stem cells are also referred to herein as “cardiomyocyte progenitor cells.”

Pluripotent stem cells are capable of developing into mature cardiomyocyte cells and at least two other types of cells. Bipotent cardiomyocyte stem cells are capable of developing into mature cardiomyocyte cells and one other type of cells, such as hematopoietic cells. Monopotent cardiomyocyte cells, i.e., cardiomyocyte progenitor cells, are capable of developing into mature cardiomyocyte cells.

By “stem cell” is meant a non-immortalized cell that possesses the capability of dividing and producing progeny that include mature, differentiated cells. The stem cells used in the present method have non-hematopoietic potential, including for example, cardiomyocyte potential or skeletal muscle potential. By “cardiomyocyte potential” is meant the ability to give rise to progeny that can differentiate into a cardiomyocyte under specific conditions. Examples of stem cells with cardiomyocyte potential include certain bone marrow cells, e.g., Oct3/4⁺ bone-marrow cells.

According to the invention, the term cardiomyocyte precursor cells always includes progenitor cells that can differentiate into cardiomyocyte progenitor cells and/or cardiomyocyte cells.

Cardiomyocyte precursor cells can be identified by factors, markers, e.g., Oct3/4, alpha-fetoprotein, Dppa 3/Stella, Dppa 4, Dppa5, FGFR1, VEGFR, FLK-1, c-Kit, as well as by alkaline phosphatase activity.

The POU (Pit-Oct-Unc)-transcription factor Oct3/4 (encoded by Pou5fl) is also known in the art as Oct 4, and is a regulator in pluripotent and germline cells, and, for example, sustains embryonic stem (ES) cell self-renewal and is a dose-dependent cell fate determinant. It is essential for the initial formation of a pluripotent founder cell population in the mammalian embryo.

The FLK-1 receptor is also known by other names, such as VEGFR-2. Human FLK-1 is sometimes referred to in the literature and herein as KDR.

High levels of c-Kit RNA transcripts are found in primary bone marrow derived mast cells and mast cell lines, while somewhat lower levels are found in melanocytes and erythroid cell lines. Hence c-Kit expression is another marker for cardiomyocyte precursor cells. The c-Kit proto-oncogene encodes a transmembrane tyrosine kinase receptor for an unidentified ligand and is a member of the colony stimulating factor-1 (CSF-1)—platelet-derived growth factor (PDGF)—kit receptor subfamily (Besmer et al., (1986) Nature 320, 415-421; Qiu et al., (1988) EMBO J. 7, 1003-1011; Yarden et al., (1987) EMBO J. 6, 3341-3351; Majumder, S., Brown, K., Qiu, F.-H. and Besmer, P. (1988) Mol. Cell. Biol. 8, 4896-4903). c-Kit is allelic with the white-spotting (W) locus of the mouse. Mutations at the W locus affect proliferation and/or migration and differentiation of germ cells, pigment cells and distinct cell populations of the hematopoietic system during development and in adult life. The W locus effects hematopoiesis through the erythroid lineages, mast cell lineages and stem cells, resulting in a macrocytic anemia which is lethal for homozygotes of the most severe W alleles, and a complete absence of connective tissue and mucosal mast cells.

A population of cardiomyocyte precursor cells can be isolated from cell sources such as bone marrow. In some embodiments, the bone marrow cells of the invention that give rise to cardiac myocytes are Oct3/4⁺ Sca1⁻CD34⁻ c-Kit^{+/-}Flk1^{+/-}FGFR^{+/-}. Bone marrow may be obtained from a mammal, such as a human patient who will undergo autologous transplantation of the collected cells. The source may be derived from an adult or from the post-natal mammal, i.e., the bone marrow cells can be senescent cells and need not be obtained from embryonic tissues. Instead, the bone marrow cells can be obtained from an older patient, even one with a vascular disease. Thus, the source of cells therefore need not be embryonic or fetal. However, the source of cells from which isolated cardiomyocyte precursor cells are derived may be any natural or non-natural mixture of cells that contain cardiomyocyte precursor cells.

Isolated cells are not necessarily pure cells; instead, isolated cells are removed from their natural source, environment or from the mammal where they naturally arose. Isolated cells can also be obtained from in vitro cultures of cell lines or from cultured embryonic cells. Cardiomyocyte precursor calls can be purified from a mixed population of cells, such as bone marrow cells, by extracting them or removing them from the bone marrow. However, no such purification is needed so long as no adverse immunological reaction will occur upon administration to a mammal. The term purified as applied to the cardiomyocyte precursor cell population utilized herein means that the population is significantly enriched in cardiomyocyte precursor cells relative to the crude population of cells from which the cardiomyocyte precursor cells are isolated.

Cardiomyocyte cell precursors can be purified, for example, from preparations of bone marrow or from in vitro-derived cells, such as those derived from allogeneic embryonic cells, nuclear transfer-derived stem cells and parthenogenetically-derived stem cells. Any available method can be used for such purification. Methods that can be employed include, for example, fluorescence-activated cell sorting (FACS) or immunomagnetic separation (for example, see Peichev et al., Blood, 2000, 95(3):952-958); and Otani et al., Nature Medicine, 2002, 8(9): 1004-1010, the contents of both of which are incorporated herein by reference in their entirety). For example, the purification procedure can lead at least to a two-fold, three-fold, five-fold, ten-fold, fifteen-fold, twenty-fold, or twenty-five fold increase in cardiomyocyte precursor cells over the total population. The purified population of cardiomyocyte precursor cells can contain at least about 15%, preferably at least 15%, at least about 20%, preferably at least 20%, at least about 25%, preferably at least 25%, at least about 35%, preferably at least 35%, or at least about 50%, preferably at least 50% of cardiomyocyte precursor cells.

The methods of the invention can also utilize cellular mixtures comprising about 30%, about 50%, about 75%, about 80%, about 85%, about 90% or about 95% of cardiomyocyte precursor cells, and preferably comprises 30%, 50%, 75%, 80%, 85%, 90% or 95% cardiomyocyte precursor cells. The methods of the invention can also utilize cell mixtures comprising 99%, 99.9% and even 100% of cardiomyocyte precursor cells. Accordingly, cell populations utilized in the invention contain significantly higher levels of cardiomyocyte precursor cells than those that exist in nature.

Cardiomyocyte precursor cells can be identified by observing their expression patterns or by contacting the cells with a molecule that binds specifically to the extracellular portion of an antigen specific for cardiomyocyte precursor cells. The binding of the cardiomyocyte precursor cells to the molecule permits the cardiomyocyte precursor cells to be sufficiently distinguished from contaminating cells that do not express the antigen to permit identification of the cardiomyocyte precursor cells from the contaminating cells.

The cells can also be purified by genetic selection techniques available in the art. For example, a nucleic acid encoding resistance to an antibiotic (such as the neomycin) can be operably linked to a nucleic acid encoding a promoter that is specifically active in an cardiomyocyte precursor to generate an expression cassette. The expression cassette can then be transfected into stem cells and the stem cells can be used to generate cardiomyocyte precursor cells that can express the neomycin resistance function. Cells that do not differentiate into cardiomyocyte precursor cells will not be resistant to neomycin because the promoter will not be active in those cells.

The molecule used to identify cardiomyocyte precursor cells can also be used to separate cardiomyocyte precursor cells from the contaminating cells. Such a molecule can be any molecule that is specifically expressed within the cardiomyocyte precursor cells or that binds specifically to an antigen that characterizes the cardiomyocyte precursor cell. The molecule can be, for example, a monoclonal antibody, a fragment of a monoclonal antibody, or, in the case of an antigen that is a receptor, the ligand of that receptor. For example, in the case of a VEGF receptor, such as FLK-1, the ligand is VEGF. Other molecules that can be used to identify and separate cardiomyocyte precursor cells from other cells include PDGF alpha receptor, VEGF-1 receptor, VEGF-2 receptor, VEGF-3 receptor, VEGF A, VEGF B, VEGF C, VEGF D, VEGF E, EGF, EGF receptor; tumor necrosis factor alpha and tumor necrosis factor receptor, and peptides discovered by phage display to specifically bind to such cells.

Either before or after the crude cell populations are purified as described above, the cells may be further enriched in precursor cells by methods known in the art.

The cardiomyocyte precursor cells can be identified within the mixture of cells obtained by exposing the cells to a molecule that binds specifically to the antigen marker characteristic of cardiomyocyte precursor cells. The molecule is preferably an antibody or a fragment of an antibody. A convenient antigen marker is Oct3/4, or a VEGF receptor, for example, a FLK-1 receptor. The cells that express the antigen marker bind to the molecule. The molecule distinguishes the bound cells from unbound cells, permitting separation and isolation. If the bound cells do not internalize the molecule, the molecule may be separated from the cell by methods known in the art. For example, antibodies may be separated from cells with a protease such as chymotrypsin.

The molecule used for isolating the purified populations of cardiomyocyte precursor cells is advantageously conjugated with labels that expedite identification and separation. Examples of such labels include magnetic beads, biotin, which may be removed by avidin or streptavidin, fluorochromes, which may be used in connection with a fluorescence-activated cell sorter, and the like.

Any technique may be used for isolation as long as the technique does not unduly harm the cardiomyocyte precursor cells. Many such methods are known in the art.

In one embodiment, the molecule is attached to a solid support. Some suitable solid supports include nitrocellulose, agarose beads, polystyrene beads, hollow fiber membranes, and plastic petri dishes. For example, the molecule can be covalently linked to Pharmacia Sepharose 6 MB macro beads. The exact conditions and duration of incubation for the solid phase-linked molecules with the crude cell mixture will depend upon several factors specific to the system employed, as is well known in the art. Cells that are bound to the molecule are removed from the cell suspension by physically separating the solid support from the cell suspension. For example, the unbound cells may be eluted or washed away with physiologic buffer after allowing sufficient time for the solid support to bind the cardiomyocyte stem cells.

The bound cells are separated from the solid phase by any appropriate method, depending mainly upon the nature of the solid phase and the molecule. For example, bound cells can be eluted from a plastic petri dish by vigorous agitation. Alternatively, bound cells can be eluted by enzymatically “nicking” or digesting an enzyme-sensitive “spacer” sequence between the solid phase and an antibody. Suitable spacer sequences bound to agarose beads are commercially available, for example, from Pharmacia.

The eluted, enriched fraction of cells may then be washed with a buffer by centrifugation and preserved in a viable state at low temperatures for later use according to conventional technology. The cells may also be used immediately, for example by being infused intravenously into a recipient.

Methods for isolating the purified populations of cardiomyocyte precursor cells are also known. Such methods include magnetic separation with antibody-coated magnetic beads, and “panning” with an antibody attached to a solid matrix. Methods for isolating the purified populations of cardiomyocyte precursor cells include general fluorescence activated cell sorting (FACS) protocols. In one embodiment, a labeled molecule is bound to the cardiomyocyte precursor cells, and the labeled cells are separated by a mechanical cell sorter that detects the presence of the label. The mechanical cell sorter is a florescence activated cell sorter (FACS) that is commercially available. Generally, the following FACS protocol is suitable for this procedure:

A Coulter Epics Eliter sorter is sterilized by running 70% ethanol through the systems. The lines are flushed with sterile distilled water.

Cells are incubated with a primary antibody diluted in Hank's balanced salt solution supplemented with 1% bovine serum albumin (HB) for 60 minutes on ice. The cells are washed with HB and incubated with a secondary antibody labeled with fluorescein isothiocyanate (FITC) for 30 minutes on ice. The secondary label binds to the primary antibody. The sorting parameters, such as baseline fluorescence, are determined with an irrelevant primary antibody. The final cell concentration is usually set at one million cells per ml.

While the cells are being labeled, a sort matrix is determined using fluorescent beads as a means of aligning the instrument.

Once the appropriate parameters are determined, the cells are sorted and collected in sterile tubes containing medium supplemented with fetal bovine serum and antibiotics, usually penicillin, streptomycin and/or gentamicin. After sorting, the cells are re-analyzed on the FACS to determine the purity of the sort.

In another embodiment, the invention is directed to isolated populations of precursor cells that express a suitable marker, for example, Oct3/4 or a VEGF receptor, such as, for example, the FLK-1 receptor. This embodiment further includes isolation of purified populations of such cells.

### Generation of Cardiomyocyte Precursor Cells of the Invention

In addition to providing methods for isolating cardiomyocyte precursor cells, as described above, the present invention provides a method for producing cells for transplantation into myocardial tissue of a mammal and a method for treating cardiac dysfunction using the cells. The method involves providing Oct3/4⁺ bone-marrow derived cardiomyocyte stem cells, culturing the cells in appropriate culture medium, for example, a medium containing growth factors such as VEGF and FGF-2, under appropriate conditions to induce or promote the cells to differentiate into cardiomyogenic cells. Cardiomyocyte precursor cells produced in this manner can be genetically modified to express a useful gene product, for example, a gene product that augments repair of cardiac injury or disease, or a gene product that prevents development of cardiac disease. The cardiomyocyte cell precursors can home to vascular tissues and provide angiogenesis (for example, in the coronary arteries of the heart), thereby restoring vascular tissues that have been injured or have become diseased.

In one embodiment of the present invention, cardiomyocyte cell precursors are isolated from a human or a non-human mammal by available methods, for example, as described above in the previous section. These cells can be genetically modified in vitro to contain a genomically integrated DNA expression construct encoding a gene that confers therapeutic effect when it is expressed by cardiomyocyte cells in the heart.

In an alternative embodiment of the invention, healthy somatic cells are isolated from a human or a non-human mammal and used for generating totipotent or pluripotent embryo-derived stem cells (e.g., embryonic stem cells). In this embodiment, the nuclei from these somatic cells are inserted into an enucleated oocyte by available procedures to generate a nuclear transfer unit that is stimulated to divide, thereby generating totipotent or pluripotent embryo-derived stem cells. The totipotent or pluripotent embryo-derived stem cells can be induced to differentiate into cardiomyocyte precursor cells, which in turn can differentiate to generate genetically modified cardiomyocyte cell precursors of the invention. Prior to nuclear transfer, the somatic cell can be genetically modified to contain a gene that confers a therapeutic effect when expressed by a cardiomyocyte, or alternatively, such modifications can be introduced in the resulting stem cells.

All types of somatic cells can be utilized as donor cells for this purpose. For example, the donor cell or donor cell nucleus can be selected from the group consisting of epithelial cells, neural cells, epidermal cells, keratinocytes, hematopoietic cells, melanocytes, chondrocytes, lymphocytes, erythrocytes, macrophages, monocytes, mononuclear cells, fibroblasts, muscle cells, skin cells, lung cells, pancreatic cells, liver cells, stomach cells, intestinal cells, heart cells, bladder cells, reproductive organ cells, urethra cell, and kidney cells.

The cardiomyocyte precursor cells, whether genetically modified or not, are then administered to a patient with a cardiac dysfunction, whereupon the cardiomyocyte precursor cells home to sites of vascular injury or areas of ischemic injury (see, for example, Asahara et al., 1997, “Isolation of putative progenitor endothelial cells for angiogenesis,” Science, 275: 964-967, the contents of which are incorporated herein by reference). After reaching the site of vascular injury the cardiomyocyte precursor cells help to prevent or repair vascular disease or vascular injury. Expression of a transgene can further enhance the therapeutic effect of these cells.

Advanced Cell Technology, Inc. and other groups have developed methods for transferring the genetic information in the nucleus of a somatic or germ cell from a child or adult into an unfertilized egg cell, and culturing the resulting cell to divide and form a blastocyst embryo having the genotype of the somatic or germ nuclear donor cell. Methods for cloning by such methods are referred to as cloning by “somatic cell nuclear transfer,” because somatic donor cells are commonly used. Methods for cloning by nuclear transfer are available, and are described, for example, in U.S. Pat. Nos. 6,235,970 (Stice et al.) and 6,147,276 (Campbell et al.), and in U.S. Pat. Nos. 5,994,619 and 6,235,969 of Stice et al., the contents of all three are incorporated herein by reference in their entirety.

Methods for human therapeutic cloning have been described. For example, methods that use nuclear transfer cloning to produce cells and tissues for transplant therapies that are histocompatible with the transplant recipient are described in U.S. Application Publication No. 20020046410 filed Mar. 5, 2001. This application also discloses assay methods for determining the immune-compatibility of cells and tissues for transplant the contents of which are incorporated herein by reference in their entirety. Similar methods are also described in U.S. Application Publication No. 20030224345 (“Screening Assays for Identifying Differentiation-Inducing Agents and Production of Differentiated Cells for Cell Therapy”), filed Aug. 26, 2002, the contents of which are also incorporated herein by reference in their entirety, which further discloses screening methods that make use of gene trapped cell lines and provide means for efficiently identifying combinations of biological, biochemical, and physical agents or conditions that induce stem cells to differentiate into cell types useful for transplant therapy. Methods for producing totipotent and pluripotent stem cells are also described in U.S. Application Publication No. 20030129745 filed Nov. 29, 2001, and International Application No. PCT/US02/22857 filed Jul. 18, 2002, which further describe methods for producing histocompatible cells and tissues for transplant by androgenesis and gynogenesis. A method for obtaining totipotent and pluripotent stem cells from embryos generated by parthenogenesis is also reported by Cibelli et al., who describe the isolation of a non-human primate stem cell line from the inner cell mass of parthenogenetic Cynomologous monkey embryos that is capable of differentiating into cell types of all three embryonic germ layers (see Science (2002) 295:819, the contents of which are incorporated herein by reference in their entirety.) The disclosures of all of the above-listed patent applications are also incorporated herein by reference in their entirety.

A general procedure for cloning by fusion of a somatic cell is provided below. The procedure is meant to be exemplary. Many variations and modifications can be made to such a procedure by one of skill in the art without deviating from the invention.

In general, oocytes are isolated from the ovaries or reproductive tract of a human or non-human mammal, matured in vitro, and stripped of cumulus cells to prepare for nuclear transfer. Alternatively, oocytes can be generated from the bone marrow-derived stem cells of the invention. Removal of the endogenous chromosomes of the oocyte is referred to as “enucleation.” Enucleation of the recipient oocyte is performed after the oocyte has attained the metaphase II stage, and can be carried out before or after nuclear transfer. Enucleation can be confirmed by visualizing chromosomal DNA in TL-HEPES medium plus Hoechst 33342 (3 μg/ml; Sigma).

Individual donor cells are placed in the perivitelline space of the recipient enucleated oocyte, and the oocyte and donor cell are fused together to form a single cell (nuclear transfer unit) e.g., by electrofusion. The nuclear transfer units are activated, and are incubated in suitable medium under conditions that promote growth of the nuclear transfer unit. During this period of incubation, the nuclear transfer units can be transferred to culture plates containing a confluent feeder layer. Feeder layers of various cell types from various species, e.g., irradiated mouse embryonic fibroblasts, that are suitable for the invention are described, for example, in U.S. Pat. No. 5,945,577, the contents of which are incorporated herein by reference in their entirety.

Genetically modified nuclei can be generated and fused with enucleated oocytes as follows. Primary cultures of somatic cells are isolated and grown in vitro using available methods. Such methods are described, for example, in U.S. Pat. No. 6,011,197 (Strelchenko et al.), and in U.S. Pat. No. 5,945,577 (Stice et al.), the contents of both of which are incorporated herein by reference in their entirety.

The somatic donor cell used for nuclear transfer to produce a nuclear transplant unit or embryo according to the present invention can be of any germ cell or somatic cell type in the body. For example, the donor cell can be a germ cell, or a somatic cell selected from the group consisting of fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, macrophages, monocytes, and mononuclear cells. The donor cell can be obtained from any organ or tissue in the body; for example, it can be a cell from an organ selected from the group consisting of liver, stomach, intestines, lung, pancreas, cornea, skin, gallbladder, ovary, testes, vasculature, brain, kidneys, urethra, bladder, and heart, or any other organ.

A general procedure for isolating primary cultures of fibroblast cells is as follows: Minced tissue is incubated overnight at 10° C. in trypsin, cells are washed and then are plated in tissue culture dishes and cultured in alpha-MEM medium (BioWhittaker, Walkersville, Md.) supplemented with 10% fetal calf serum (FCS) (Hyclone, Logen, Utah), penicillin (100 μl/ml) and streptomycin (50 μl/ml). The fibroblast cells can be isolated at virtually any time in development, ranging from approximately post embryonic disc stage through adult life of the animal (for example, for bovine, from day 12 to 15 after fertilization to 10 to 15 years of age).

A general procedure for stably introducing a genetic expression construct into the genomic DNA of the cultured fibroblasts by electroporation is described below. Other available transfection methods, such as microinjection or lipofection can also be used to introduce heterologous DNA into the cells.

Culture plates containing propagating fibroblast cells are incubated in trypsin EDTA solution (0.05% trypsin/0.02% EDTA; GIBCO, Grand Island, N.Y.) until the cells are in a single cell suspension. The cells are spun down at 500×g and re-suspended at a density of about 5 million cells per ml with phosphate buffered saline (PBS). A vector or nucleic acid construct containing an expression cassette encoding the gene product of interest is added to the cells in the electroporation chamber. After providing a standard electroporation pulse, the fibroblast cells are transferred back into growth medium (alpha-MEM medium (BioWhittaker, Walkersville, Md.) supplemented with 10% fetal calf serum (FCS) (Hyclone, Logen, Utah), penicillin (100 μl/ml) and streptomycin (50 μl/ml)).

The day after electroporation, attached fibroblast cells are selected for stable integration of the vector or nucleic acid construct by culturing them for up to 15 days in growth medium containing a selective agent that will select for cells having the vector or nucleic acid construct. At the end of the selection period, colonies of stable transgenic cells are present. Each colony is propagated independently of the others. Transgenic fibroblast cells can be further tested for expression of the gene product of interest, and genomic integration of the expression construct can be confirmed by available methods; e.g., by PCR amplification and analysis by agarose gel electrophoresis.

Stably transfected fibroblast cells are used as nuclear donors in the nuclear transfer (NT) procedure. Procedures for cloning by nuclear transfer are available in the art. For example, methods for cloning by somatic cell nuclear transfer are described in detail in U.S. Pat. No. 6,147,276 (Campbell et al.), and in co-owned and co-assigned U.S. Pat. Nos. 5,945,577 and 6,235,969 of Stice et al.

In general, oocytes are isolated from the ovaries or reproductive tract of a human or non-human mammal and are matured in vitro. The oocytes are stripped of cumulus cells to prepare for nuclear transfer. Enucleation of the recipient oocyte is performed after the oocyte has attained the metaphase II stage, and can be carried out before or after nuclear transfer. Individual donor cells (fibroblasts) are then placed in the perivitelline space of the recipient oocyte, and the oocyte and donor cell are fused together to form a single cell (an nuclear transfer unit) using electrofusion techniques; e.g., by applying a single one fusion pulse consisting of 120 V for 15 μsec to the nuclear transfer unit in a 500 μm gap chamber. The nuclear transfer units are then incubated in suitable medium.

A variety of different procedures for artificially activating oocytes are available and have been described. Following activation, the nuclear transfer units are washed and cultured under conditions that promote growth of the nuclear transfer unit to have from 2 to about 400 cells. During this time, the nuclear transfer units can be transferred to well plates containing a confluent feeder layer; e.g., a feeder layer of mouse embryonic fibroblasts. Feeder layers of various cell types from various species that are suitable for the invention are described, for example, in U.S. Pat. No. 5,945,577. Multicellular non-human nuclear transfer units produced in this manner can be transferred into recipient non-human females of the same species as the donor nucleus and recipient oocyte, for development into transgenic non-human mammals. Alternatively, the nuclear transfer units can be incubated until they reach the blastocyst stage, and the inner cell mass (ICM) cells of these nuclear transfer units can be isolated and cultured in the presence or absence of a feeder layer to generate pluripotent or totipotent embryonic stem cells. These stem cells can then be differentiated to generate downstream cultured stem cells such as the mesodermal precursors to hemangioblasts.

Multicellular non-human nuclear transfer units produced in this manner can be transferred as embryos into recipient non-human females of the same species as the donor nucleus and recipient oocyte, for development into transgenic non-human mammals. Alternatively, the nuclear transfer units can be incubated in vitro until they reach the blastocyst stage, and the inner cell mass (ICM) cells of these nuclear transfer units can be isolated and cultured in the presence or absence of a feeder layer to generate pluripotent or totipotent embryo-derived stem cells, including totipotent embryonic stem cells.

Generating Embryoid Bodies (EBs) from the Oct3/4⁺ Bone Marrow-derived Precursor Cells of the Invention

Embryoid bodies (EBs) form spontaneously in Oct3/4(+) bone-marrow derived cell cultures (see Example 2, below) that have been maintained in the presence of VEGF and FGF, as described herein. Additional factors can be added to enhance or direct this process, including, but not limited to, retinoic acid, dimethylsulfoxide (DMSO), cAMP elevators such as forskolin, isobutylmethylxanthine, and dibutryl cAMP, cytokines such as basic fibroblast growth factor, epidermal growth factor, platelet derived growth factor (PDGF and PDGF-AA) nerve growth factor, T3, sonic hedgehog (Shh or N-Terminal fragment), ciliary neurotrophic factor (CNTF), erythropoietin (EPO) and bone morphogenic factors. Additional growth factors can also be added to the culture. “Growth factor” as used herein refers to a substance that is effective to promote the growth of EBs that is not otherwise a component of the growth medium. Such substances include, but are not limited to, cytokines, chemokines, small molecules, neutralizing antibodies, and proteins. Growth factors also include intercellular signaling polypeptides that control both the development and maintenance of cells, and the form and function of tissues.

Cultures are monitored daily for embryoid body formation.

Generating Germ Cells from the Oct3/4⁺ Bone Marrow-Derived Precursor Cells of the Invention

The continuation of mammalian species requires the formation and development of the sexually dimorphic germ cells. Cultured embryonic stem cells are generally considered pluripotent, rather than totipotent, because of the failure to detect germline cells under differentiating conditions. However, cultured mouse embryonic stem cells have been reported to develop into oogonia that enter meiosis, recruit adjacent cells to form follicle-like structures, and later develop into blastocysts (Hübner et al., Science, 300:1251 (2003)). Accordingly, the invention contemplates germ cells or gamete-like cells derived from the Oct3/4 bone marrow-derived stem cells of the invention. Such gamete-like cells include cells having characteristics of primordial germ cells, spermatocytes, oocytes, and the like.

The term “oocyte” is used to describe the mature animal ovum, which is the final product of oogenesis. The phrase “oocyte-like cell” broadly refers to any cell having characteristics of an oocyte or a precursor form of an oocyte, i.e., an oogonium, a primary oocyte or a secondary oocyte. Oogenesis begins with the formation of primordial germ cells (PGC's), a source of adult germ cells. Primordial germ cells arise in the extra-embryonic tissues of the yolk sac and allantois, migrate into the hindgut epithelium and along the dorsal mesentary of the genital ridges and finally arrive in the primitive gonad. The PGC's undergo approximately 7 to 8 mitotic divisions during migration until 2 to 3 days after arrival in the ovary and are converted to oogonia, which are connected by intercellular bridges (cell syncytium) and begin actively dividing. Oogonia become oocytes once they cease mitosis and enter meiosis. Meiosis continues until oocytes reach the dictyate stage of the first meiotic prophase, which is at or shortly after parturition in most species. During this stage, oocytes will undergo a period of extensive growth and discontinue meiosis until the gonadotropin surge at ovulation. It is here that meiosis resumes and continues until oocytes are arrested at metaphase II (unfertilized oocytes). Meiotic reduction also begins as evidenced by first polar body extrusion. Oocytes will then remain at this stage until fertilization or parthenogenetic activation, at which time meiosis is completed and the second polar body is extruded.

The most dramatic aspect of oocyte growth is the 300-fold increase in size to become one of the largest cells in the body. During oocyte growth, some distinct structural changes occur. These include an increase in the diameter of the nucleus (or germinal vesicle; GV) as well as a marked decrease in the nuclear to cytoplasm ratio, enlargement and a change from a diffuse, granular to a dense, fibrillar network of nucleoli, increase in the number of mitochondria as well as a change from elongated mitochondria with transverse cristae to round mitochondria with columnar cristae, a change in Golgi membranes from flat stacks of arched lamellae with no vacuoles to swollen stacks of lamellae with many vacuoles, appearance of cortical granules, appearance and growth of the zona pellucida, increase in the number of ribosomes, and appearance of cytoplasmic lattices.

Biochemical changes also occur during oocyte growth. An extremely large amount of total ribonucleic acid (RNA; 200-fold levels in somatic cells; and protein (50-fold levels in somatic cells) synthesis and storage is present in growing murine oocytes. These accumulate primarily because cytokinesis does not occur, although the concentration of total RNA and protein are not different from somatic cells. Some specific proteins that are synthesized during murine oocyte growth are mitochondrial and ribosomal proteins, zona pellucida glycoproteins, histones, tubulin, actin, calmodulin, lactate dehydrogenase, creatine kinase and glucose-6-phosphate dehydrogenase. Changes in specific gene expression during oocyte growth have been reported for murine oocytes. These include presence of oct-3 messenger RNA (mRNA) in growing oocytes, an increase in number of c-kit transcripts, increase in transcription of m-ZP3 and unusually high levels of lactate dehydrogenase activity in oocytes prior to meiotic maturation as well as numerous others.

Meiotic maturation is defined as the progression from the dictyate stage of the first meiotic prophase to metaphase II. Oocytes acquire meiotic competence by obtaining the ability to progress from GV breakdown to metaphase I and then obtaining the ability to progress from metaphase I to metaphase II. Porcine oocytes from follicles with an average diameter of 3 mm have attained meiotic competence. Meiotic maturation is composed of a number of structural changes. Probably the most obvious structural change is GV (or nuclear) breakdown. This is very evident in murine oocytes; however, this can only be seen via a nuclear stain in porcine oocytes. The next sequence of landmarks include chromosome condensation (transition from diffuse dictyate-stage to V-shaped, telocentric bivalent chromosomes), spindle formation and first polar body extrusion. Throughout these events, a number of alterations in microtubule and microfilament structure occur. Other biochemical changes occur during meiotic maturation including a dramatic decrease in RNA levels, a decrease in intracellular methionine levels and a decrease in protein synthesis.

Certain regulatory molecules are also involved in meiotic maturation. Factors suggested to inhibit GV breakdown are cyclic adenosine monophosphate and regulators of its intracellular levels, calcium, calmodulin, steroids, gonadotropins, purines, protein inhibitors and intercellular communication between cumulus cells and the oocyte. Two hypotheses for the resumption of meiosis by luteinizing hormone (LH) at ovulation are loss of inhibitory input and positive stimuli. The loss of inhibitory input hypothesis suggests that inhibitory substances (e.g., cyclic adenosine monophosphate) produced by granulosa or cumulus cells maintain meiotic arrest and the LH surge may terminate communication between the follicle granulosa cells and cumulus cells or between cumulus cells and the oocyte resulting in the absence of this inhibitory stimulus to the oocyte. The positive stimuli theory suggests that LH may induce production of a substance (calcium, adenosine triphosphate, pyruvate) from granulosa or cumulus cells that directly causes the oocyte to resume meiosis.

Dramatic decreases in tubulin, actin, histone, ribosomal protein, lactate dehydrogenase and zona pellucida glycoprotein synthesis rates occur as well as phosphorylation changes in cell cycle control proteins. Changes in specific gene expression during meiotic maturation have been reported for murine oocytes. These include a decrease in c-mos transcription between metaphase I and II, presence of oct-3 mRNA in ovulated oocytes, a dramatic drop in m-ZP3 RNA levels at ovulation, appearance of tissue-type plasminogen activator transcripts following GV breakdown and a sharp decrease in lactate dehydrogenase levels during meiotic maturation.

An important cytoplasmic factor involved in meiotic maturation is a protein called MPF. Maturation (M-phase, mitosis, meiosis) promoting factor is ubiquitous to all dividing yeast, invertebrate, amphibian and mammalian cells and it controls the transition from the G2 to mitosis phases of the cell cycle. Two subunits form the MPF complex including a 34 kilodalton (kD) catalytic subunit (p34cdc2; a protein kinase) and a 45 kD regulatory subunit (cyclin B). Levels of p34cdc2 are constant while cyclin levels fluctuate throughout the cell cycle. Immature oocytes contain a precursor to MPF which is the inactive form and dephosphorylation of p34cdc2 at tyrosine and threonine residues results in the active state of MPF, which is required for GV breakdown. At the end of metaphase I (prior to first polar body extrusion), the cyclins are degraded rendering the MPF complex inactive. New cyclins are synthesized and MPF becomes highly active during metaphase II. Levels of MPF remain high during metaphase II due to a protein called cytostatic factor (CSF). This protein contains products of the c-mos (pp 39mos; a 39 kD phosphoprotein) and cdk-2 (cyclin-dependent kinase 2) genes and appears to act by preventing cyclin degradation. Upon oocyte activation, CSF is destroyed by a protease that is activated by the release of Ca2⁺ ions and MPF levels drop allowing meiosis completion and pronuclear formation. Examination of histone H1 kinase is used as a reflection of MPF activity because p34cdc2 has been shown to phosphorylate histone H1 in vitro. These phosphorylation events have been used as a biochemical assay for the estimation of p34cdc2 activity.

The successful in vitro development of the oocyte has become much more important in recent years with the advances in molecular biology and an increased push for the production of transgenic animals. The present invention provides a method of preserving fertility, for example, in subjects who require chemotherapy. As disclosed herein, a subpopulation of bone marrow-derived cells express Oct3/4, a marker associated with an undifferentiated/pluripotential state. Oct3/4 is a POU-domain transcription factor associated with pluripotent stem cell capacity and is strongly expressed in female germ cells. Thus, in one embodiment of the method, a germ cell or a gamete-like cell, for example, an oocyte-like cell, is generated in vitro using the bone-marrow derived Oct3/4⁺ precursor cells of the invention. In another embodiment, an oocyte-like cell is generated that is Oct3/4⁺/Sca1⁻/CD34⁻/cKit⁺/Flk1⁺/FGFR⁺. The in vitro generated oocyte can then develop into a structure resembling a blastocyst in, for example, a petri dish without being fertilized (parthenogenesis).

Differentiation of Cardiomyocyte Precursor Cells into Cardiomyocytes

In addition to the above, the present invention provides a method to generate a cardiomyocyte, i.e., a differentiated cardiomyocyte, from the cardiomyocyte precursor cells of the invention. Using methods provided herein, totipotent and pluripotent stem cells derived from Oct3/4⁺ bone-marrow stem cells can be cultured under conditions that direct or allow differentiation into a variety of partially and fully differentiated cardiomyocytes. The adjective “differentiated” is a relative term. A “differentiated cell” is a cell that has progressed further down the developmental pathway than the cell it is being compared with. Thus, cardiomyocyte precursor cells of the invention can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

Cell fate during development can be defined by transcription factors that act as molecular switches to activate or repress specific gene expression programs. Phenotypic features of the precursor cells, cardiomyogenic cells and differentiated cardiomyocytes of the invention can be studied using characteristics, i.e., markers, which are known to the art. For example, commonly analyzed markers utilized to characterize stem cells such as Embryonic Stem (ES) cells, Embryonic Carcinoma (EC) cells, and Embryonic Germ (EG) cells can be used to evaluate the purity and stage of differentiation of the cells of the invention. These markers include, but are not limited to, the glycolipid surface Stage Specific Embryonic Antigens (SSEA-1, SSEA-3, SSEA-4), the keratan sulphate-related antigens Tra-1-60 and Tra-1-81 and the transcription factor Octamer-4 (Oct-4), which is also know as Oct-3/4.

The carbohydrate antigen SSEA-1 appears during late cleavage stages of mouse embryos. It is strongly expressed by undifferentiated, murine ES cells. Upon differentiation, murine ES cells are characterized by the loss of SSEA-1 expression and may be accompanied, in some instances, by the appearance of SSEA-3 and SSEA-43. In contrast, human ES and EC cells typically express SSEA-3 and SSEA-4 but not SSEA-1, while their differentiation is characterized by down regulation of SSEA-3 and SSEA-4 and an up regulation of SSEA-14, 5. Undifferentiated, human ES cells also express the antigens, TRA-1-60 and TRA-1-816.

During embryogenesis, expression of Oct-4, which is a member of the POU family of transcription factors, is limited to pluripotent cells of the inner cell mass (ICM) that contribute to the formation of all fetal cell types. As development proceeds, Oct-4 expression is restricted to cells of the germline. This relationship between Oct-4 and pluripotency has seen this transcription factor emerge as an important marker of pluripotent stem cells. All mammalian pluripotent stem cells that include Embryonic Stem (ES), Embryonic Carcinoma (EC) and Embryonic Germ (EG) cells express Oct-4. Significantly, this expression decreases following stem cell differentiation.

An additional characteristic of undifferentiated Embryonic Stem (ES) cells is the expression of high levels of Alkaline Phosphatase (AP) on their cell surface. Because this expression decreases following stem cell differentiation, the assessment of AP expression-serves as a method for analyzing stem cell differentiation, including expression of GATA-4, MLC-2a, MLC-2v, ANF, and Nkx2.5.

Differentiation of the bone-marrow cells can be monitored, and cardiomyogenic cells with certain phenotypic features harvested.

Accordingly, stem cells isolated or generated as described herein can readily be differentiated into cardiomyocyte precursor cells.

As discussed herein, to promote cardiomyocytes formation from the Oct3/4⁺ bone marrow cells, the bone marrow cells can be cultured in culture medium that has a sufficient amount of a growth factor, such as vascular endothelial growth factor (VEGF) and fibroblast growth factor-2 (FGF-2) for a time and under conditions sufficient to generate cardiomyocytes. In one embodiment, the culture medium employed does not contain insulin-like growth factor-1 (IGF-1). VEGF and FGF-2 are commercially available and can be obtained, for example, from R&D Systems.

A sufficient amount of vascular endothelial growth factor (VEGF) is about 0.001 ng/mL to about 10 mg/mL, or about 0.01 ng/mL to about 1 mg/mL, or about 0.1 ng/mL to about 100 ng/mL or about 1 ng/mL to about 100 ng/mL vascular endothelial growth factor (VEGF). In certain embodiments, bone marrow cells were successfully treated with vascular endothelial growth factor (VEGF) at concentrations of about 10 ng/mL.

A sufficient amount of fibroblast growth factor-2 (FGF-2) is about 0.001 ng/mL to about 10 mg/mL, or about 0.01 ng/mL to about 1 mg/mL, or about 0.1 ng/mL to about 100 ng/mL or about 1 ng/mL to about 100 ng/mL fibroblast growth factor-2 (FGF-2). In certain embodiments, bone marrow cells were successfully treated with fibroblast growth factor-basic at concentrations of about 5 ng/mL.

The time used to generate cardiomyocytes from bone marrow by VEGF and FGF-2 treatment can vary. For example, culturing bone marrow cells in the presence of VEGF and FGF-2 for a time period of a few days (about 3 days) to several weeks (about 5 weeks) can lead to cardiomyocytes generation from bone marrow cells. In experiments described herein, bone marrow cells were successfully cultured for about 1 week in order to facilitate cardiomyocyte formation.

Conditions required for culturing bone marrow cells to generate cardiomyocytes comprise the conditions normally employed for culturing mammalian cells in vitro. Inclusion of heparin (at about 50 μg/mL) helps support the generation of cardiomyocytes from bone marrow cells in vitro.

### Syngeneic Cardiomyocyte Precursor Cells

In a useful embodiment of the invention, bone marrow cells are taken from a patient with a cardiac dysfunction. These syngeneic cells can be treated to generate useful cells for treatment of cardiac dysfunction. For example, bone marrow cells can be cultured with VEGF and FGF-2 to generate syngeneic cardiomyocytes that can be re-administered to the patient. Such bone marrow cells can also be genetically modified to contain a gene that confers a therapeutic effect. The genetically modified bone marrow cells can then be administered to the patient. Such syngeneic stem cells can be induced to differentiate into cardiomyocyte precursor cells that can give rise to genetically modified cardiomyocyte cells in vivo. The genetically modified cardiomyocyte cell precursors are then administered to a patient as an autologous transplant, whereupon the cardiomyocyte cells derived therefrom home to sites of cardiac angiogenesis or vessel repair. Since the transplanted bone marrow cells and cardiomyocyte cell precursors are syngeneic with the patient, they are histocompatible and do not elicit an immune response, unless such a response is elicited by expression of the transgene.

An alternative embodiment of the invention that does not use nuclear transfer-derived cells can be practiced as follows:

Cardiomyocyte cell precursors can also be isolated from the patient, genetically modified in vitro to contain a gene that confers a therapeutic effect, and are reintroduced to the patient as described in PCT Publication WO 99/37751 by Shahin Rafii, Larry White and Malcolm A. Moore, and U.S. Pat. No. 5,980,887 (Isner et al.), the contents of which are incorporated herein by reference in their entirety. In brief, a sample of bone marrow is collected from the patient. A population of cells positive for antigens specific for cardiomyocyte cell precursors is then isolated. For example, the remaining cells can be treated with fluorochrome labeled antibodies to the antigens specific for cardiomyocyte cell precursors and isolated by Fluorescence Activated Cell Sorting (FACS). Alternatively, cardiomyocyte cell precursors can be isolated by magnetic beads coated with the above antibodies to the above antigens, as is available in the art. Once purified, the population of cell precursors are cultured in vitro in suitable medium and the cells are genetically modified using methods known in the art. Following genetic modification, the cells are intravenously reintroduced to the patient.

### Allogeneic, HLA-Matched Cardiomyocyte Cell Precursors

Banks of bone marrow cells or of pre-made embryonic stem cell lines can be isolated, where the bone marrow cells or embryonic stem cell lines are each homozygous for at least one MHC gene. Such banks of cells serve as an alternative to using nuclear transfer cloning to produce syngeneic embryonic stem cells de novo and inducing these to differentiate into the required cells for every patient that is in need of therapeutic transplant. However, homozygous embryos generated in vitro or in vivo can serve as a source of homozygous MHC stem cells.

The MHC genes of humans are also referred to as HLA (human leukocyte antigen) genes or alleles. Such MHC and HLA genes are highly polymorphic, and banks of different embryonic stem cell lines and different bone marrow isolates with different MHC and HLA genes will include a large number of different embryonic stem cell lines. Once such banks of bone marrow isolates or embryonic stem cells with homozygous MHC alleles are produced, it is possible to provide a patient in need of cell transplant with MHC-matched cells and tissues by selecting and/or expanding a line of bone marrow cells of embryonic stem cells that has MHC allele(s) that match one of those of the patient. The bone marrow or embryonic stem cells can be treated with PDGF AB or other agents to differentiate into the type of cells that the patient requires. Methods for preparing a bank of embryonic stem cell lines that are homozygous for the MHC alleles, and for using these to provide MHC-matched cells and tissues for transplantation therapies are described in co-pending U.S. Provisional Patent Application No. 60/382,616, entitled, “A Bank of Nuclear Transfer-Generated Stem Cells for Transplantation Having Homozygous MHC Alleles, and Methods for Making and Using Such a Stem Cell Bank, filed May 24, 2002, the disclosure of which is incorporated herein by reference in its entirety.

Therefore, in another useful embodiment of the invention, the bone marrow and nuclear donor cells that are genetically modified are not obtained from the patient. Instead, they are taken from a person who has HLA alleles that match those of the patient. More simply, the bone marrow or nuclear donor cells are taken from a person who has homozygous HLA alleles that match at least one HLA allele of the patient. A bank of samples of viable nuclear donor cells, each sample made up of cells having homozygous HLA alleles that match an HLA allele found in the population, is prepared and maintained for practicing this embodiment. See U.S. Provisional Patent Application No. 60/382,616. As described above for syngeneic transplant therapy, genetically modified, HLA-matched cardiomyocyte cell precursors produced by the invention are administered to a patient as a heterologous transplant, to give rise to cardiomyocyte cells that home to and incorporate into the tumor vasculature to disrupt or inhibit tumor angiogenesis. Since the transplanted cardiomyocyte cell precursors are HLA-matched to the patient, they are partially histocompatible with the patient, and so do not elicit the strong rejection response that would be elicited by a completely allogeneic transplant.

In an alternative embodiment, cells of one or more of the established human embryonic stem cell lines are genetically modified, and available methods are used to induce the genetically modified embryonic stem cells to differentiate into cardiomyocyte cell precursors. These cardiomyocyte cell precursors can then give rise to genetically modified cardiomyocyte cells that confer a therapeutic effect when recruited into a site of vascular injury or ischemic myocardium. Alternatively, cardiomyocyte cell precursors can be isolated directly from a young person other than the patient and when appropriate to the needs of that patient, genetically modified, to confer a therapeutic effect. The cardiomyocyte precursors obtained from differentiating embryonic stem cells or directly from a person other than the patient can then be transplanted into the patient.

### Modification of Cardiomyocyte Precursor Cells and Cardiomyocytes of the Invention

As discussed herein, transgenic cells of the invention that are genetically modified to contain a stably integrated gene that is expressed in cardiomyocytes cells and that confers a therapeutic effect are obtained by methods available in the art. Recombinant expression vectors are made and introduced into the cells using standard techniques, e.g., electroporation, lipid-mediated transfection, or calcium-phosphate mediated transfection, and cells containing stably integrated expression constructs are selected or otherwise identified, also using standard techniques known in the art. Methods for making recombinant DNA expression constructs, introducing them into eukaryotic cells, and identifying cells in which the expression construct is stably integrated and efficiently expressed, are described, for example, in Sambrook, et al., Molecular Cloning: A Laboratory Manual, 2d Edition, Cold Spring Harbor Laboratory Press (1989); Sambrook, et al., Molecular Cloning: A Laboratory Manual, 3^{rd} Edition, Cold Spring Harbor Laboratory Press (2001). Such methods useful for practicing the present invention are also described, for example, in U.S. Pat. No. 5,980,887.

There are a variety of different types of genes that confer a therapeutic effect when expressed in cardiomyocyte cells in sites of cardiac disease or dysfunction, e.g., vascular injury or I-ischemic myocardium. For example, cardiomyocyte precursor cells of the invention can be used to administer therapeutic agents such as enzymes, peptides and/or proteins with biological activity, nucleic acids or genes that encode therapeutic polypeptides, expression vectors or other nucleic acid constructs, for example, naked plasmid DNAs, any vector carrying one or more genes, any sense or anti-sense RNA, or any ribozyme. Nucleic acids encoding such therapeutic agents are introduced into cardiomyocyte precursor cells based upon their ability to optimally treat one or more vascular conditions. For example, the cardiomyocyte precursor cell can be designed to help control, diminish or otherwise facilitate improved arterial blood flow in the region of the atherosclerotic lesion.

Such therapeutic agents include, for example, thrombolytic agents such as streptokinase, tissue plasminogen activator, plasmin and urokinase, anti-thrombotic agents such as tissue factor protease inhibitors (TFPI), anti-inflammatory agents, metalloproteinase inhibitors, nematode-extracted anticoagulant proteins (NAPs) and the like. Other examples of therapeutic agents that can be expressed in the cardiomyocyte precursor cells of the invention include the following: agents that modulate lipid levels (for example, HMG-CoA reductase inhibitors, thyromimetics, fibrates, agonists of peroxisome proliferator-activated receptors (PPAR) (including PPAR-alpha, PPAR-gamma and/or PPAR-delta)); agents that modulate oxidative processes such as modifiers of reactive oxygen species; agents that modulate insulin resistance or glucose metabolism (e.g. agonists of PPAR-alpha, PPAR-gamma and/or PPAR-delta, modifiers of DPP-IV, and modifiers of glucocorticoid receptors); agents that modulate expression of receptors or adhesion molecules or integrins on endothelial cells or smooth muscle cells in any vascular location; agents that modulate the activity of endothelial cells or smooth muscle cells in any vascular location; agents that modulate inflammation associated receptors (e.g. chemokine receptors, RAGE, toll-like receptors, angiotensin receptors, TGF receptors, interleukin receptors, TNF receptors, C-reactive protein receptors, and other receptors involved in inflammatory signaling pathways including the activation of NF-kb); agents that modulate proliferation, apoptosis or necrosis of endothelial cells, vascular smooth muscle, lymphocytes, monocytes, and neutrophils that adhere to or within the vessel; agents that modulate production, degradation, or cross-linking of any extracellular matrix proteins (e.g. collagen, elastin, and proteoglycans); agents that modulate activation, secretion or lipid loading of any cell type within mammalian vessels; agents that modulate the activation or proliferation of dendritic cells within mammalian vessels; and agents that modulate the activation or adhesion of platelets within blood vessels.

Hence, the cardiomyocyte precursor and other cells of the invention can be modified to express a therapeutic agent such as those described herein. Such genetic modifications can be performed by procedures available to one of skill in the art. For example, a nucleic acid encoding the therapeutic agent can be placed within an expression cassette or expression vector, and the cassette or vector can be introduced into the cell. The expression cassette can be placed within a vector to generate an expression vector.

Any vector that can replicate in a selected cell can be utilized in the invention. In general, the vector is an expression vector that provides the nucleic acid segments needed for expression of the therapeutic agent polypeptides. Various vectors are publicly available. The vector may, for example, be in the form of a plasmid, cosmid, viral particle, or phage. Vector components generally include, but are not limited to, one or more of a signal sequence, an origin of replication, one or more marker genes, an enhancer element, a promoter, and a transcription termination sequence.

The therapeutic agent nucleic acid sequences may be inserted into the vector by a variety of procedures. In general, DNA is inserted into an appropriate restriction endonuclease site(s) using techniques known in the art. See generally, Sambrook et al., 1989, Molecular Cloning, A Laboratory Manual, 2d ed., Cold Spring Harbor Laboratory Press, Cold Spring Harbor, N.Y.; Sambrook and Russell, Molecular Cloning: A Laboratory Manual, 3rd edition (Jan. 15, 2001) Cold Spring Harbor Laboratory Press, ISBN: 0879695765; Ausubel et al., Current Protocols in Molecular Biology, Green Publishing Associates and Wiley Interscience, NY (1989)). Construction of suitable expression vectors containing a therapeutic agent can employ standard ligation techniques that are known to the skilled artisan.

The expression cassette or vector of the invention includes a promoter. A promoter is a nucleotide sequence that controls expression of an operably linked nucleic acid sequence by providing a recognition site for RNA polymerase, and possibly other factors, required for proper transcription. A promoter includes a minimal promoter, consisting only of all basal elements needed for transcription initiation, such as a TATA-box and/or other sequences that serve to specify the site of transcription initiation. Any promoter able to direct transcription of an RNA encoding the selected therapeutic agent may be used. Accordingly, many promoters may be included within the expression cassette or vector of the invention. Some useful promoters include constitutive promoters, inducible promoters, regulated promoters, cell specific promoters, viral promoters, and synthetic promoters. A promoter may be obtained from a variety of different sources. For example, a promoter may be derived entirely from a native gene, be composed of different elements derived from different promoters found in nature, or be composed of nucleic acid sequences that are entirely synthetic. A promoter may be derived from many different types of organisms and tailored for use within a given cell, for example, a cardiomyocyte precursor cell.

Many mammalian promoters are known in the art that may be used in conjunction with the expression cassette of the invention. Mammalian promoters often have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter may also contain an upstream promoter element, usually located within 100 to 200 bp upstream of the TATA box. An upstream promoter element determines the rate at which transcription is initiated and can act in either orientation (Sambrook et al., “Expression of Cloned Genes in Mammalian Cells”, in: Molecular Cloning: A Laboratory Manual, 2nd ed., 1989).

Mammalian viral genes are often highly expressed and have a broad host range; therefore sequences encoding mammalian viral genes often provide useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallothionein gene, also provide useful promoter sequences. Expression may be either constitutive or regulated.

A mammalian promoter may also be associated with an enhancer. The presence of an enhancer will usually increase transcription from an associated promoter. An enhancer is a regulatory DNA sequence that can stimulate transcription up to 1000-fold when linked to homologous or heterologous promoters, with synthesis beginning at the normal RNA start site. Enhancers are active when they are placed upstream or downstream from the transcription initiation site, in either normal or flipped orientation, or at a distance of more than 1000 nucleotides from the promoter. (Maniatis et al., Science, 236:1237 (1987); Alberts et al., Molecular Biology of the Cell, 2nd ed., 1989)). Enhancer elements derived from viruses are often times useful, because they usually have a broad host range. Examples include the SV40 early gene enhancer (Dijkema et al., EMBO J., 4:761 (1985) and the enhancer/promoters derived from the long terminal repeat (LTR) of the Rous Sarcoma Virus (Gorman et al., Proc. Natl. Acad. Sci. USA, 79:6777 (1982b)) and from human cytomegalovirus (Boshart et al., Cell, 41: 521 (1985)). Additionally, some enhancers are regulatable and become active only in the presence of an inducer, such as a hormone or metal ion (Sassone-Corsi and Borelli, Trends Genet., 2:215 (1986); Maniatis et al., Science, 236:1237 (1987)).

It is understood that many promoters and associated regulatory elements may be used within the expression cassette of the invention to transcribe an encoded polypeptide. The promoters described above are provided merely as examples and are not to be considered as a complete list of promoters that are included within the scope of the invention.

The expression cassettes and vectors of the invention may contain a nucleic acid sequence for increasing the translation efficiency of an mRNA encoding a therapeutic agent of the invention. Such increased translation serves to increase production of the therapeutic agent. Because eucaryotic mRNA does not contain a Shine-Dalgarno sequence, the selection of the translational start codon is usually determined by its proximity to the cap at the 5′ end of an mRNA. However, the nucleotides immediately surrounding the start codon in eucaryotic mRNA influence the efficiency of translation. Accordingly, one skilled in the art can determine what nucleic acid sequences will increase translation of a polypeptide encoded by the expression cassettes and vectors of the invention.

Termination sequences can also be included in the cassettes and vectors of the invention. Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation (Birnstiel et al., Cell, 41:349 (1985); Proudfoot and Whitelaw, “Termination and 3′ end processing of eukaryotic RNA”, in: Transcription and Splicing (eds. B. D. Hames and D. M. Glover) 1988; Proudfoot, Trends Biochem. Sci., 14:105 (1989)). These sequences direct the transcription of an mRNA that can be translated into the polypeptide encoded by the DNA. Examples of transcription terminator/polyadenylation signals include those derived from SV40 (Sambrook et al., “Expression of cloned genes in cultured mammalian cells”, in: Molecular Cloning: A Laboratory Manual, 1989).

As indicated above, nucleic acids encoding the therapeutic agents can be inserted into any convenient vector. Vectors that may be used include, but are not limited to, those that can be replicated in prokaryotes and eukaryotes. For example, vectors may be used that are replicated in bacteria, yeast, insect cells, and mammalian cells. Examples of vectors include plasmids, phagemids, bacteriophages, viruses, cosmids, and F-factors. However, specific vectors may be used for specific cells types. Additionally, shuttle vectors may be used for cloning and replication in more than one cell type. Such shuttle vectors are known in the art. The nucleic acid constructs or libraries may be carried extrachromosomally within a host cell or may be integrated into a host cell chromosome. Numerous examples of vectors are known in the art and are commercially available. (Sambrook and Russell, Molecular Cloning: A Laboratory Manual, 3rd edition (Jan. 15, 2001) Cold Spring Harbor Laboratory Press, ISBN: 087-9695765; New England Biolab, Beverly, Mass.; Stratagene, La Jolla, Calif.; Promega, Madison, Wis.; ATCC, Rockville, Md.; CLONTECH, Palo Alto, Calif.; Invitrogen, Carlabad, Calif.; Origene, Rockville, Md.; Sigma, St. Louis, Mo.; Pharmacia, Peapack, N.J.; USB, Cleveland, Ohio). These vectors also provide many promoters and other regulatory elements that those of skill in the art may include within the nucleic acid constructs of the invention through use of known recombinant techniques.

A nucleic acid construct, or an expression vector can therefore be inserted into any mammalian vector that is known in the art or that is commercially available, for example, as provided by CLONTECH (Carlsbad, Calif.), Promega (Madison, Wis.), or Invitrogen (Carlsbad, Calif.). Such vectors may contain additional elements such as enhancers and introns having functional splice donor and acceptor sites. Nucleic acid constructs may be maintained extrachromosomally or may integrate in the chromosomal DNA of a host cell. Mammalian vectors include those derived from animal viruses, which require trans-acting factors to replicate. For example, vectors containing the replication systems of papovaviruses, such as SV40 (Gluzman, Cell, 23:175 (1981)) or polyomaviruses, replicate to extremely high copy number in the presence of the appropriate viral T antigen. Additional examples of mammalian vectors include those derived from bovine papillomavirus and Epstein-Barr virus. Additionally, the vector may have two replication systems, thus allowing it to be maintained, for example, in mammalian cells for expression and in a prokaryotic host for cloning and amplification. Examples of such mammalian-bacteria shuttle vectors include pMT2 (Kaufman et al., Mol. Cell. Biol. 9:946 (1989)) and pHEBO (Shimizu et al., Mol. Cell. Biol., 6:1074 (1986)).

The invention is directed to cells that express a heterologous protein or overexpress a native protein, and nucleic acids or expression vector encoding such a heterologous or native protein. Such cells may be used for treating and preventing vascular conditions, as described herein.

Methods for introduction of heterologous polynucleotides into mammalian cells are known in the art and include lipid-mediated transfection, dextran-mediated transfection, calcium phosphate precipitation, polybrene-mediated transfection, protoplast fusion, electroporation, encapsulation of -the polynucleotide(s) in liposomes, biollistics, and direct microinjection of the DNA into nuclei. The choice of method depends on the cell being transformed as certain transformation methods are more efficient with one type of cell than another. (Felgner et al., Proc. Natl. Acad. Sci., 84:7413 (1987); Felgner et al., J. Biol. Chem., 269:2550 (1994); Graham and van der E b, Virology, 52:456 (1973); Vaheri and Pagano, Virology, 27:434 (1965); Neuman et al., EMBO J., 1:841 (1982); Zimmerman, Biochem. Biophys. Acta., 694:227 (1982); Sanford et al., Methods Enzymol., 217:483 (1993); Kawai and Nishizawa, Mol. Cell. Biol. 4:1172 (1984); Chaney et al., Somat. Cell Mol. Genet., 12:237 (1986); Aubin et al., Methods Mol. Biol., 62:319 (1997)). In addition, many commercial kits and reagents for transfection of eukaryotic cells are available.

Following transformation or transfection of a nucleic acid into a cell, the cell may be selected for the presence of the nucleic acid through use of a selectable marker. A selectable marker is generally encoded on the nucleic acid being introduced into the recipient cell. However, co-transfection of selectable marker can also be used during introduction of nucleic acid into a host cell. Selectable markers that can be expressed in the recipient host cell may include, but are not limited to, genes that render the recipient host cell resistant to drugs such as actinomycin C₁, actinomycin D, amphotericin, ampicillin, bleomycin, carbenicillin, chloramphenicol, geneticin, gentamycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. (Davies et al., Ann. Rev. Microbiol., 32: 469 (1978)). Selectable markers may also include biosynthetic genes, such as those in the histidine, tryptophan, and leucine biosynthetic pathways. Upon transfection or transformation of a cell, the cell is placed into contact with an appropriate selection agent.

Accordingly, the invention provides methods for generating and using genetically modified cardiomyocyte precursor cells that can express useful therapeutic agents.

### Suicide Genes for Eliminating Grafted Cardiomyocyte Cells

The invention is also directed to genetically-modified cardiomyocyte cell precursors that express a selectable suicide gene, such as thymidine kinase (TK), which allows negative selection of grafted cells upon completion of treatment or in the event of undesired complications. TK-expressing cells can be negatively selected by the administration of gancyclovir according to methodology known in the art. Alternatively, the cardiomyocyte cell precursors can be genetically-modified to express cytosine deaminase, which causes the cells to die in the presence of added 5-fluorocytosine. The expressed gene can be lethal as a toxin or lytic agent.

Cardiomyocyte precursor cells and other cells can be genetically modified to express such “suicide genes” by available recombinant techniques, for example, as described herein.

### Therapeutic Uses of the Cardiomyocyte Precursor Cells of the Invention

The diseases and conditions treated by the present invention are cardiac diseases of mammals. The word mammal means any mammal. Some examples of mammals include, for example, pet animals, such as dogs and cats; farm animals, such as pigs, cattle, sheep, and goats; laboratory animals, such as mice and rats; primates, such as monkeys, apes, and chimpanzees; and humans. In some embodiments, humans are preferably treated by the methods of the invention.

The present invention also provides for cardiomyocytes derived from bone-marrow cells that may be used therapeutically for treatment of various diseases associated with cardiac dysfunction. As discussed herein, the bone marrow cells from which the cardiomyocyte precursor cells and cardiomyocytes are generated can be obtained from an older patient, even one with a vascular disease. Thus, after re-introducing the cells to the patient, no tissue rejection or other immunological problems will arise. Hence, the inventive methods avoid side effects and other complications.

The term “cardiac disease,” “cardiac condition” or “cardiac dysfunction” as used herein refers to diseases that result from any impairment in the heart's pumping function, e.g., a disease characterized by insufficient cardiac function. This includes, for example, impairments in contractility, impairments in ability to relax (sometimes referred to as diastolic dysfunction), abnormal or improper functioning of the heart's valves, diseases of the heart muscle (sometimes referred to as cardiomyopathy), diseases such as angina and myocardial ischemia and infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart (for example, atrial septal defect). For further discussion, see Braunwald, Heart Disease: a Textbook of Cardiovascular Medicine, 5th edition, W B Saunders Company, Philadelphia Pa. (1997). The term “cardiomyopathy” refers to any disease or dysfunction of the myocardium (heart muscle) in which the heart is abnormally enlarged, thickened and/or stiffened. As a result, the heart muscle's ability to pump blood is usually weakened. The disease or disorder can be, for example, inflammatory, metabolic, toxic, infiltrative, fibroplastic, hematological, genetic, or unknown in origin. There are two general types of cardiomyopathies: ischemic (resulting from a lack of oxygen) and nonischemic. Other diseases include congenital heart disease that is a heart-related problem that is present since birth and often as the heart is forming even before birth or diseases that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease.

In some embodiments, the cardiac disease or condition arises from damaged myocardium. As used herein “damaged myocardium” refers to myocardial cells that have been exposed to ischemic conditions. These ischemic conditions may be caused by a myocardial infarction, or other cardiovascular disease. The lack of oxygen causes the death of the cells in the surrounding area, leaving an infarct that can eventually scar.

As an example, myocardium is treated with the methods and compositions of the invention before damage occurs (e.g., when damage is suspected of occurring) or as quickly as possible after damage occurs. Hence, the methods and compositions of the invention are advantageously employed on heart tissues that are in danger of ischemia, heart attack or loss of blood flow. The methods and compositions of the invention are also advantageously employed on recently damaged myocardium and on not so recently damaged myocardium.

As used herein “recently damaged myocardium” refers to myocardium that has been damaged within about one week, and preferably within one week of treatment being started. In a preferred embodiment, the myocardium has been damaged within about three days, and preferably within three days of the start of treatment. In a further preferred embodiment, the myocardium has been damaged within about twelve hours, and preferably within twelve hours of the start of treatment.

Further examples of cardiac dysfunction, vascular conditions or vascular disease to which the methods of the invention apply are those in which the vasculature of the affected tissue or system is altered in some way such that blood flow to the tissue or system is reduced or in danger of being reduced. Vascular, circulatory or hypoxic conditions to which the methods of the invention apply are those associated with, but not limited to, maternal hypoxia (e.g., placental hypoxia, preclampsia), abnormal pregnancy, peripheral vascular disease (e.g., arteriosclerosis), transplant accelerated arteriosclerosis, deep vein thrombosis, erectile dysfunction, cancers, renal failure, stroke, heart disease, sleep apnea, hypoxia during sleep, female sexual dysfunction, fetal hypoxia, smoking, anemia, hypovolemia, vascular or circulatory conditions which increase risk of metastasis or tumor progression, hemorrhage, hypertension, diabetes, vasculopathologies, surgery (e.g., per-surgical hypoxia, post-operative hypoxia), Raynaud's disease, endothelial dysfunction, regional perfusion deficits (e.g., limb, gut, renal ischemia), myocardial infarction, stroke, thrombosis, frost bite, decubitus ulcers, asphyxiation, poisoning (e.g., carbon monoxide, heavy metal), altitude sickness, pulmonary hypertension, sudden infant death syndrome (SIDS), asthma, chronic obstructive pulmonary disease (COPD), congenital circulatory abnormalities (e.g., Tetralogy of Fallot) and Erythroblastosis (blue baby syndrome). In particular embodiments, the invention is a method of treating loss of circulation or cardiac dysfunction in an individual.

The methods and compositions of the invention can be used to prevent or to treat these conditions. These methods involve administering an effective amount of cardiomyocyte precursor cells, for example, stem cells, or cardiomyocytes, e.g., differentiated cells. Such an amount is effective when it stimulates the generation of cardiomyocytes or restores cardiac function in a tissue.

Any method known to one of skill in the art may be utilized to assess cardiac function. In one embodiment, the beating rate of a cardiomyocyte may also be assayed to identify agents that increase or decrease beating. One method for assessing the beating rate is to observe beating under a microscope. Agents that can be screened in this manner include inotropic drugs, such as sympathomimetic agents.

The bone-marrow derived cardiomyocyte precursor cells, i.e., stem cells and/or differentiated cardiomyocytes of the invention may be administered and/or transplanted to a subject suffering from cardiac dysfunction or cardiac disease in any fashion known to the art. For example, cardiomyocyte precursor cells may be administered in any manner used by one of skill in the art to introduce the cells into the vascular system of the host. The cells may be introduced into a specific site in the vascular system to optimize delivery to a site that is known to have a vascular condition or disease. Such local delivery may avoid stimulation of inappropriate vascularization, for example, within a tumor that may be present in the mammal. However, cardiomyocyte precursor cells can find their way to diseased vascular tissues, so local administration may not be needed.

Cardiomyocyte precursor cells and/or bone marrow cells may be administered by intravascular, intravenous, intraarterial, intraperitoneal, intraventricular infusion, infusion catheter, balloon catheter, bolus injection, direct application to tissue surfaces during surgery, or other convenient routes. The cells can be washed after collection, cultured in an appropriate medium to insure their viability and to enhance their numbers. Prior to administration, the cells can also be cultured in the presence of growth factors such as VEGF, FGF-2, PDGF (e.g. PDGF AB), G-CSF, GM-CSF, VEGF, SCF (c-kit ligand), bFGF, chemokines such as SDF-1, or interleukins such as interleukins 1 and 8. Before administration, the cells can be washed again, for example, in buffered physiological saline.

The volume of cells that is injected and the concentration of cells in the transplanted solution depend on the site of administration, the vascular disease, and the species of the host. Preferably about one-half to about five microliters is injected at a time. The number of cells injected can vary, for example, about 10² to about 10¹⁰ or about 10⁴ to about 10⁹ cells can be injected at one time. While a single injection may be sufficient, multiple injections may also be used for prevention or treatment of vascular diseases.

Compositions of the invention may also contain a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. The use of such media and agents for delivering cells is well known in the art. Except insofar as any conventional media or agent is incompatible with the cells or polypeptides provided herein, use thereof in the compositions is contemplated. Supplementary active compounds can also be incorporated into the compositions.

A pharmaceutical composition of the invention is formulated to be compatible with its intended route of administration. Examples of routes of administration include intravenous, intraarterial, intracoronary, parenteral, subcutaneous, subdermal, or subcutaneous. Solutions or suspensions used for such administration can include other components such as sterile diluents like water for dilution, saline solutions, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The composition can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions to accompany the cellular suspensions can be prepared by incorporating an active compound (e.g., VEGF, FGF-2) in the required amount in an appropriate solvent with a selected combination of ingredients, followed by filter sterilization. Generally, dispersions are prepared by incorporating an active compound into a sterile vehicle that contains a basic dispersion medium and the required other ingredients from those enumerated above. In the case of sterile powders for the preparation of sterile injectable solutions, methods of preparation are vacuum drying and freeze-drying that yield a powder of the active ingredient plus any additional desired ingredient from a previously sterile-filtered solution thereof.

It is especially advantageous to formulate the cells and/or compositions in dosage unit form for ease of administration and uniformity of dosage. Dosage unit form as used herein refers to physically discrete units suited as unitary dosages for the subject to be treated. Each unit can then contain a predetermined quantity of the cardiomyocyte precursor cells and/or bone marrow cells and other components calculated to produce the desired therapeutic effect in association with the required pharmaceutical carrier.

The cellular preparations and pharmaceutical compositions can be included in a kit, e.g., in a container, pack, or dispenser together with instructions for administration.

### Screening Agents Using Cells of Invention

Methods are also provided for screening agents that affect cardiomyocyte differentiation or function. According to one method, a population of cardiomyocytes may be produced as described herein, a population of cells is contacted with an agent of interest, and the effect of the agent on the cell population is then assayed. For example, the effect on differentiation, survival, proliferation, or function of the cells may then be assessed. Such screening assays may involve the measurement of calcium transients. In one embodiment calcium imaging is used to measure calcium transients. For example, ratiometric dyes, such as fura-2, fluo-3, or fluo-4 are used to measure intracelluar calcium concentration. The relative calcium levels in a population of cells treated with a ratiometric dye can be visualized using a fluorescent microscope or a confocal microscope. In other embodiments, the membrane potential across the cell membrane is monitored to assess calcium transients. For example, a voltage clamp may be used. In this method, an intracellular microelectrode is inserted into the cardiomyocyte. In one embodiment, calcium transients can be seen before observable contractions of the cardiomyocytes. In other embodiments calcium transients are seen either during, or after, observable contractions of cardiomyocytes. In another embodiment the cells are cultured in the presence of conditions wherein the cells do not beat, such as in the presence of a calcium chelator (e.g., EDTA or EGTA) and the calcium transients are measured.

The following examples are intended to illustrate the invention and should not be interpreted to limit it in any nature.

### EXAMPLE 1

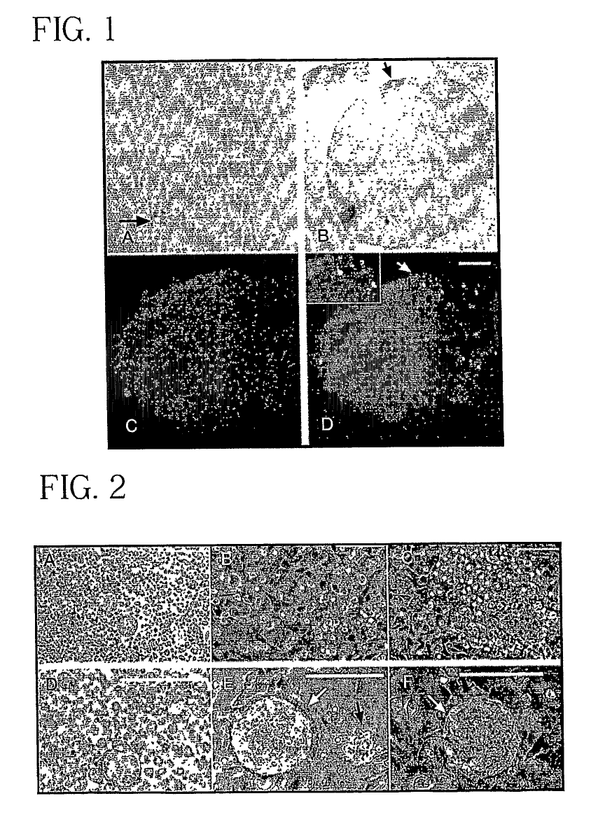

Co-culture experiments using day 14 fetal hearts and Rosa26 total bone marrow (TBM) cells (FIG. 1 A-B) showed that TBM cells engrafted into heart tissue by day 2 (FIG. 1A), that engraftment was maximal at day 7, and that engrafted cells tended to localize close to vascular structures (FIG. 1B). Co-culture experiments with 5-chloromethyl fluorescein diacetate (CMFDA) labeled wild-type TBM (FIG. 1 C-D) were used to confirm engraftment of TBM cells into fetal hearts. These data suggest that local environmental cues are important in recruitment and/or differentiation of bone marrow cells in the heart.

Recently, a novel system was developed to induce cardiomyocyte differentiation by culturing TBM in the presence of VEGF and FGF-2 (Xaymardan et al., Circ. Res., 94:E39-45 (2004)) suggesting that TBM cell cultures may retain/obtain the plasticity to generate an array of end organ cell types. Phenotypic analysis indicated that, alongside differentiation of stromal cells (FIG. 2B) and haematopoietic cells (FIG. 2C), bone marrow stem cells differentiated into contracting cardiomyocytes by day 7. Furthermore, under the same culture conditions, spheroidal bodies (FIG. 2A, FIG. 2D), similar to embryoid-body-like structures previously described in the culture of embryonic stem cells. The TBM cells developed into larger embryoid-body-like clusters (40-250 uM; 148±85 cells), with a frequency of 10.7±5.2/animal, at day 7. The percentage of beating clusters observed in total bone marrow cultures at day 7 was 32 8±25.9% (FIG. 2E), similar to the percentage observed for embryonic stem cell-derived embryoid bodies (Lake et al., 2000). The number of embryonic stem cell-like clusters decreased during culture. By day 14, the number of clusters was 5.8±5.7/animal. Beating structures appeared at day 6-7 and were no longer observed after day 12.

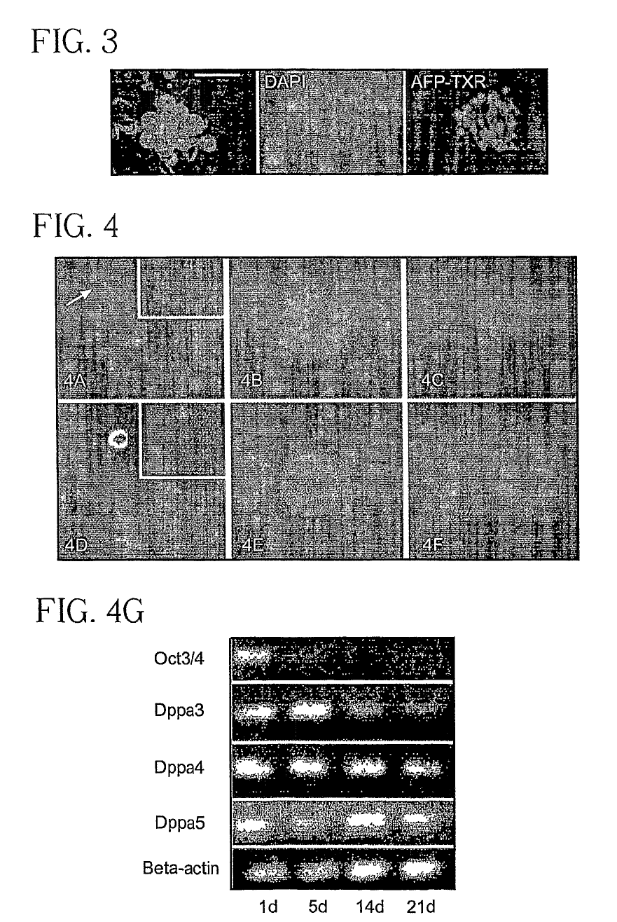

Immunocytochemical analysis revealed that alpha-fetoprotein (AFP) was expressed in the bone marrow-derived embryoid-like bodies (FIG. 3). AFP is a biomarker of primordial endoderm expressed by embryonic stem (ES) cell-derived embryoid bodies (EBs), which confirmed that embryoid bodies were formed from bone marrow cells (FIG. 3). These results suggest that cytokine and cell to cell interactions play pivotal roles in the development and differentiation of adult stem cells from the bone marrow, similar to what has been observed for ES cells (Cheng et al., 2003 and Amit et al., 2004). Conditions permissive for the development of EB-like structures might also enable differentiation of pluripotent bone marrow cells into cell types of uncommitted/non-mesodermal origin.

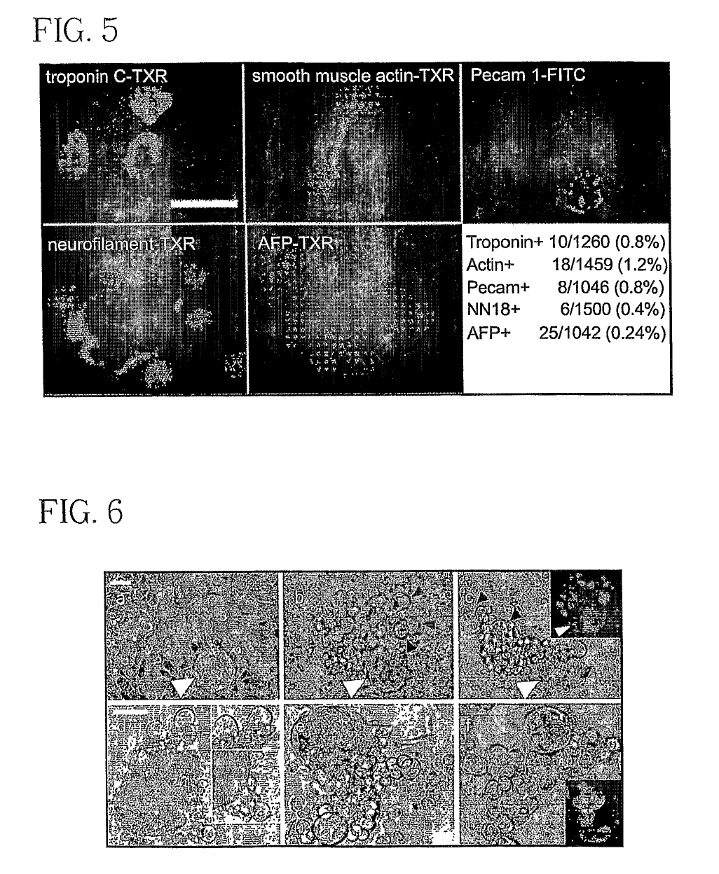

Molecular analysis of the cultures demonstrated that the TBM expressed the pluripotent stem cell (specific) gene OCT-4 (FIG. 4). These studies demonstrated that the TBM cells were composed of a subpopulation of OCT-4 positive cells. While the overall expression of OCT-4 decreased with culture time, the EB-like structures were composed of OCT-4 cells, suggesting that the cluster of cells could give rise to pluripotent stem cells that generate end organ cells and tissues from adult TBM cells. Indeed, FIG. 5 demonstrates the potential of the culture conditions disclosed herein to give rise to all three primitive cell lineages in vitro.

### EXAMPLE 2

### Cardiomyocyte Precursors for Enhancing and Restoring Cardiac Function

This Example illustrates that the induction of functional cardiac myocytes from murine bone marrow cells in culture is mediated by the differentiation of a sub-population of cells expressing Oct-3/4, a marker associated with an undifferentiated/pluripotent state. These Oct-3/4⁺ cells can spontaneously form embryoid body-like aggregates that generate clusters of cardiac myocytes, independent of the need for pre-existing cardiac muscle cells. Bone marrow-derived Oct-3/4⁺ cells were also able to contribute to in vivo regeneration of infarcted heart tissue, confirming their potential for future clinical applications.

### Materials and Methods

Animals: Experiments described herein employed 3 month old C57B1/6 female mice and were performed in compliance with the institutional Animal Care and use Committee of Weill Medical College of Cornell University.

Bone marrow cell culture: Whole bone marrow cells were isolated as previously described (Xaymardan et al., 2004) and cultured for 14 days in 12 well plates, at a concentration of 1.5×10⁶/cm², in Iscove's Modified Dulbecco's Medium (IMDM, Gibco) supplemented with 10% Fetal Bovine Serum (FBS), 1% penicillin/streptomycin, 50 μg/ml heparin sodium salt (Sigma), 10 ng/ml VEGF (recombinant human, R&D Systems) and 5 ng/ml FGF-2 (recombinant human, R&D Systems). These conditions induce cardiomyogenic differentiation of bone marrow cells, with expression of connexin 43, β-MHC and α-MHC by day 14 of culture (Xaymardan et al, 2004). Cells were monitored every 24 hours for activity and formation of bone marrow aggregates and appearance of spontaneous beating activity (4 replicates, 3 mice/replicate). Purified cell aggregates were cultured on fibronectin-coated wells (0.1 ug/ml) until day-21. Purified aggregates were also collected on cytospin slides and immunostained for troponin-T.

ES-cell culture: The ES-cell line ES-D3 was purchased from ATCC and sub-cultured on following the company guidelines. In some cases, feeder layers were employed. For differentiation (EB formation), ES-cells were cultured in bone marrow culture medium for 2 days in hanging drops, and then transferred for 14 days to cell culture dishes for differentiation.

Whole mount immunostaining for aggregates and cardiomyocyte clusters: Cells were centrifuged at 80×g for 10 minutes on cytospin slides at a concentration of 2×10⁴ cells/slide. Alternatively, clusters of spontaneously beating cells and cell aggregates at different stages of development (diameter=40-200 μM, cell number=148±85 cells/aggregate) were collected by microdissection using a 150-250 μm diameter micropipette and processed in 40 μl microdrops. Immunolabelling of the cell aggregates and clusters was performed by indirect immunofluorescence as previously described by Wreggett et al. (1994) with the following primary antibodies: anti-Oct-3/4 (rabbit), anti-Flk-1 (rabbit), anti-c-Kit (rabbit), anti-Sca1 (goat), anti-CD34 (biotinylated mouse), anti-FGFR-1 (biotinylated mouse, Chemicon International), anti-PDGFRα (rabbit), anti-AFP (goat) and anti-cardiac troponin-T (goat), not cross reactive with slow and fast skeletal muscle troponin-T. A biotinylated mouse monoclonal anti-Oct-3/4 was used for double staining with anti-c-Kit and anti-Flk-1 antibodies. Samples were then incubated with the following fluorochrome-conjugated secondary antibodies: fluoresceinisothyocynate (FITC)-anti-rabbit IgG, FITC-avidin (Vector Laboratories), Texas red (TXR)-avidin (Vector Laboratories), TXR-anti-goat IgG. Triple staining with anti-Oct-3/4 (rabbit), anti-c-Kit (rabbit) and anti-Sca1 (goat) was carried out using the Zenon Tricolor Rabbit IgG Labeling kit (Molecular Probes) and Alexa Fluor 350-anti-goat (Molecular probes). Triple staining with anti-Oct-3/4 (rabbit), anti-Flk-1 (goat) and anti-FGFR-1 (biotinylated mouse) was followed by incubation with Alexa Fluor 350-anti-goat, TXR-Avidin and FITC-anti-rabbit secondary antibodies. Samples were mounted with Vectashield mounting medium with or without the DNA dye 4′,6-diamino-2-phenylindole (DAPI; Vector, Laboratories). Mouse antibodies were biotinylated before use (InnoGenex). All antibodies were purchased from Santa Cruz, unless otherwise stated, and were used at a final concentration of 2-5 μg/ml. Incubation with primary antibodies was omitted in controls. Alkaline phosphatase (AP) activity was assayed with the AP specific chromogenic BCIP/NBT (InnoGenex). AP negative control samples were incubated at 70° C. for 30 min. Quantification was performed in duplicates (10 aggregates or 8 fields/8000 total cells/2 cytospin slides).

In some experiments, staining for Oct3/4 was performed using a protocol specific for nuclear antigens (Wregget et al., 1994). Specimens were incubated overnight at 4° C. with rabbit polyclonal anti-Oct3/4 antibodies or biotinylated mouse anti-Oct3/4 antibodies. In other experiments, staining with the biotinylated mouse anti-Oct3/4 antibody was followed by incubation for one hour at room temperature with polyclonal rabbit anti-Fetal Liver Kinase 1 (Flk1), anti-c-Kit, or polyclonal goat anti-Sca1 antibodies. Staining with the rabbit polyclonal anti-Oct3/4 antibody was followed by incubation for one hour at room temperature with a biotinylated mouse anti-Fibroblast Growth Factor Receptor antibody (FGFR-1; Chemicon International) or CD34. The same protocol was used for intracytoplasmic antigens. Samples were incubated overnight at 4° C. with a goat antibody recognizing either AFP or cardiac troponin-T, the latter were not cross-reactive with slow and fast skeletal muscle troponin-T.

When co-staining for Flk1 and FGFR-1 in aggregates, samples were fixed for 20 minutes with 2% PFA, washed with PBS, and blocked with a solution of 1% BSA in PBS and 0.03% Tween-20. Samples were stained overnight at 4° C. with a rabbit polyclonal anti-Flk1 antibody followed by 1 hour incubation at room temperature with biotinylated mouse anti-FGFR-1 antibody. After incubation with the primary antibodies, all samples were washed with TBS or PBS with 0.03% tween-20 (washing buffer), and incubated for 1 hour at room temperature with secondary antibody solutions containing 5 μg/ml fluoresceinisothyocynate (FITC)-conjugated donkey anti-rabbit IgG or FITC-conjugated avidin (Vector Laboratories) with or without Texas red (TXR)-conjugated avidin (Vector Laboratories), or TXR-conjugated donkey anti-goat IgG. Incubations with primary and secondary antibodies were followed by washes with Phosphate Buffer Solution (PBS) with 0.03% Tween-20. The specimens were finally mounted with vectashield mounting medium with the DNA dye 4′,6-diamino-2-phenylindole (DAPI; Vector Laboratories). Mouse antibodies were biotinylated before use (Innogenex, mouse on mouse kit). All antibodies were purchased from Santa Cruz unless otherwise stated and were used at a final concentration of 5 μg/ml. Incubation with primary antibodies was omitted in controls.

For detection of endogenous alkaline phosphatase (AP) activity, aggregates were fixed with 4% paraformaldehyde, permeabilized at 25° C. for 12 minutes with 0.2% Triton-X 100 in PBS, and incubated for 3 minutes with the AP specific chromogen BCIP/NBT (InnoGenex). Negative controls were incubated at 70° C. for 30 minutes before detection of AP activity.

RNA extraction, RT-PCR and quantitative-PCR: RNA was isolated from ovaries and livers by homogenizing 100 mg tissue in 1 ml trizol (Invitrogen), followed by phenol chloroform extraction and isopropyl alcohol precipitation. The yield of RNA collected was 1.5 μg/mg ovarian tissue, and 3.5 μg/mg liver tissue. Using a RNeasy Mini kit (Qiagen), RNA was also isolated from (i) bone marrow cell aggregates collected by microdissection (yielding 1.5 μg-2 μg RNA/20-30 aggregates; each aggregate contained approximately 3,000 to 5,000 cells generated from material collected from 6 different animals); and (ii) cell samples (yielding 15 μg RNA/10⁷ cells). To remove contaminating genomic DNA, RNA samples were incubated 30 minutes at 37° C. with RQ1 RNase-free DNase (Promega) at a concentration of 1 U/μg RNA.

RNA samples were processed with a two-step approach involving reverse transcription (Omniscript Reverse Transcriptase, Qiagen) followed by polymerase chain reaction using 0.5 μg template cDNA, Taq polymerase (HotStarTaq, Qiagen) and 1 μM of each set of the following primers designed to recognize sequences at the exon-intron boundary:

Oct3/4
(F) 5′TGTGGACCTCAGGTTGGACT3′ (SEQ ID NO:1),
(R) 5′CTTCTGCAGGGCTTTCATGT3′ (SEQ ID NO:2) (201
bp) using conditions 54° C., 38 cycles;
Dppa3
(F) 5′CTTTCCCAAGAGAAGGGTCC3′ (SEQ ID NO:3),
(R) 5′TGCAGAGACATCTGAATGGC3′ (SEQ ID NO:4) (149
bp) using conditions 54° C., 33 cycles;
Dppa4
(F) 5′TTCTGGATGAGAAAGGCACC3′ (SEQ ID NO:5),
(R) 5′TGCCCCAAGTGTGTTCATAA3′ (SEQ ID NO:6) (186
bp) using conditions 54° C., 33 cycles;
α-MHC
(F) ACCTGACCCAACTCCAGACA3′ (SEQ ID NO:7),
(R) 5′TCCTTCTTCAGCTCCTCAGC3′ (SEQ ID NO:8) (117
BP) using conditions 62° C., 38 cycles;
β-MHC
(F) 5′GCCAACACCAACCTGTCCAAGTTC3′ (SEQ ID NO:9),
(R) 5′TGCAAAGGCTCCAGGTCTGAGGGC3′ (SEQ ID NO:10)
(203 bp) using conditions 64° C., 38 cycles;
β-actin
(F) 5′CTGCCTGACGGCCAAGTCATCAC3′ (SEQ ID NO:11),
(R) 5′GTCAACGTCACACTTCATGATGG3′ (SEQ ID NO:12)
(141 bp) using conditions 54° C., 30 cycles;
and
PDGFB
(F) CCTTCCTCTCTGCTGCTACC-3′ (SEQ ID NO:13),
(R) 5′TCATGTTCAAGTCCAGCTCA (SEQ ID NO:14) using
conditions 54° C., 30 cycles.

PCR was conducted as follows: 1 cycle at 95° C. for 15 minutes; 30-38 (Oct3/4 and ∝ MHC) cycles at 94° C. for 30 seconds; annealing for 30 seconds at 62° C. for α-MHC; 64° C. for β-MHC, or 54° C.; 1 cycle at 72° C. for 90 seconds; followed by 1 cycle at 72° C. for 10 minutes. Amplified products were visualized by electrophoresis on 2% agarose gels. Quantitative PCR was carried out under the same cycle conditions, using a Sybr Green Master Mix (Applied Biosystems). Controls included samples processed without the reverse transcription step and samples with no template DNA. All experiments were carried out in triplicates.

Rat Myocardial Infarction (MI) model: A rat Ml model previously described by Xaymardan et al. (J Exp Med. 199, 797-804 (2004)) was used to study the ability of donor bone marrow-derived of Oct-3/4⁺ cells to participate in the regeneration of the infarcted heart tissue. An anti-apoptotic growth factor combination consisting of PDGF-AB, VEGF and Angiogenin-2 (100 ng each/50 ul PBS) was injected at the time of occlusion. Growth factor injection was omitted in controls. Donor bone marrow cells were labeled with 10 uM 5-chloromethyl-fluorescein diacetate (CMFDA, Molecular Probes) resuspended at a concentration of 5×10⁶cells/50 μl PBS and injected intramyocardially in the infarction area 15 min before coronary occlusion. Rats were sacrificed and hearts were excised and processed for immunohistochemistry 4 days after ligation.

Immunohistochemistry: Frozen heart sections were fixed in 4% PFA, incubated with anti-Oct-3/4 (rabbit; Santa Cruz Biotechnology) and anti-troponin-T (goat) primary antibodies and with Alexa Fluor 633-anti-rabbit (Molecular Probes) and rhodamine-anti-goat (Santa Cruz Biotechnology) secondary antibodies. Counts were performed in the peri-infarction tissue using a LSM 510 confocal set-up and image acquisition software (Zeiss; 63x objective; n=10 fields/heart, 3 hearts per condition).

### Results and Discussion

In order to define the mechanisms mediating the generation of bone marrow-derived cardiac myocytes, total bone marrow cells were cultured under cardiomyogenic conditions described by Xaymarden et al. (Cir. Res. 94: E39-E45 (2004)) and were visually monitored daily to identify the source of spontaneously contracting cells. Whole bone marrow cells, with no immunoselection, adhesion, or gradient centrifugation steps, were cultured under conditions that support the development of stromal cell monolayers with the ability to support stem cell self-renewal and preserve cell interactions normally found in the bone marrow microenvironment. (Cheng et al., 2003; D'Ippolito et al., 2004). Culture media was supplemented with fibroblast growth factor (FGF)-2 and vascular endothelial growth factor (VEGF). FGF-2 and VEGF are both required to support differentiation of bone marrow cells into functional cardiac myocytes (Xaymardan et al. 2004).

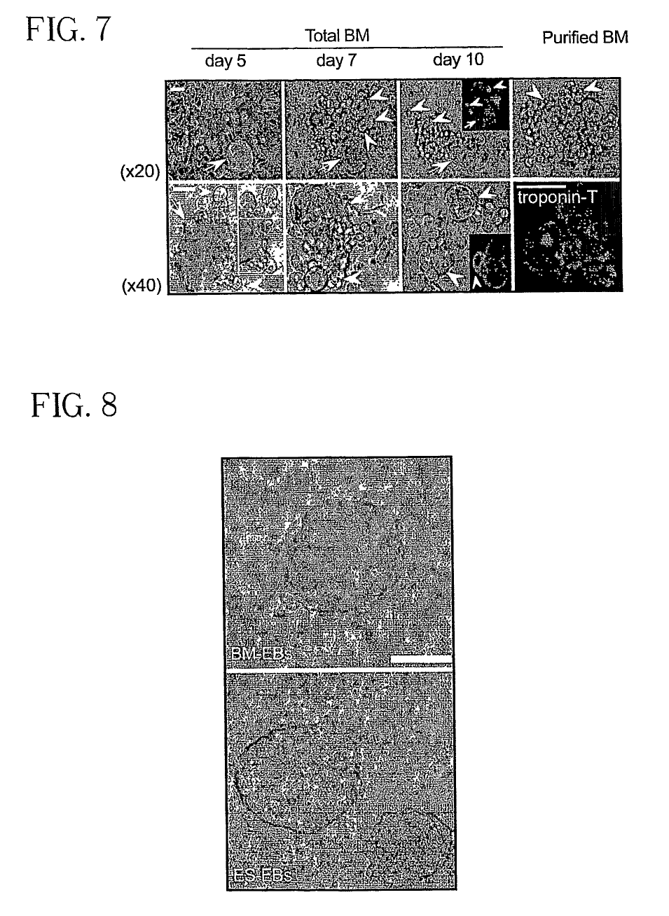

Spherical cell aggregates were first observed at day 3-5 at the onset of stroma development (FIGS. 6A, 6D, 7A, 7E). Approximately, 6.0±1.9 bone marrow cell aggregates formed per animal at day-5. By culture day 7, the number of aggregates had increased (10.7±1.9 aggregates/animal) and clusters of spontaneous contracting cells were observed at the periphery of 32.9±10.6% of these aggregates (FIGS. 6B, 7B). These spontaneously contacting cells were identified as cardiac myocytes, not only by their spontaneous chronotropic activity, but also by their positive immunostaining for cardiac troponin-T (FIGS. 7C and 7G, inserts day-10; 28.6±4.5% of cells). By day 10, the contracting cell clusters (9.3±2.6 aggregates/animal) had become larger and started detaching ((FIGS. 6C, 6F, 7C and 7G). Contracting cell clusters and/or contracting cells were observed in suspension until at least day 14. By day 10, the percentage of aggregates with contracting activity decreased to 15.9±6.8% (FIGS. 6C, 6F). This pattern of spontaneous chronotropic activity was comparable to that observed during mouse embryonic stem (ES)-cell cardiomyogenesis from ES cell-derived embryoid bodies (EBs). Autonomous beating, which is associated with cardiac differentiation, is observed between day 9 and 16 in 15-25% of the cells present in the outgrowths and in 30-80% of ES-cells embryoid bodies (EBs) (Pesce et al., 1999).

Cardiomyogenic potential of isolated bone marrow-derived cell aggregates was confirmed in culture. Bone marrow-cell aggregates selected at day-5 and cultured on fibronectin were able to generate clusters of contracting cells at day-14, as observed in 29.1% (7/24) of the cell aggregates. Contracting clusters were isolated by microdissection and immunostained of for cardiac troponin-T (FIGS. 6C, 6F). Cytospin slides prepared at day-21 confirmed expression of troponin-T in 31.3% (67/214) of the cells obtained from purified cell-aggregate cultures (FIGS. 7C, 7G and 7H). In conjunction with their chronotropic function, such troponin expression confirmed cardiac lineage of the bone marrow-derived cells.

Thus, temporal observation of cell structure, function and expression revealed that cardiac myocytes arise from spherical cellular aggregates originating from whole bone marrow-cell cultures.

Efficiency of cardiac differentiation in the purified aggregate cultures was comparable to that observed in whole bone marrow cell cultures, though the onset of chronotropic activity and expression of cardiac markers was delayed by approximately one week (FIGS. 7D and 7H). These data indicate that the stroma, though not necessary for cardiac differentiation, may play an important support role in cardiac cell development.

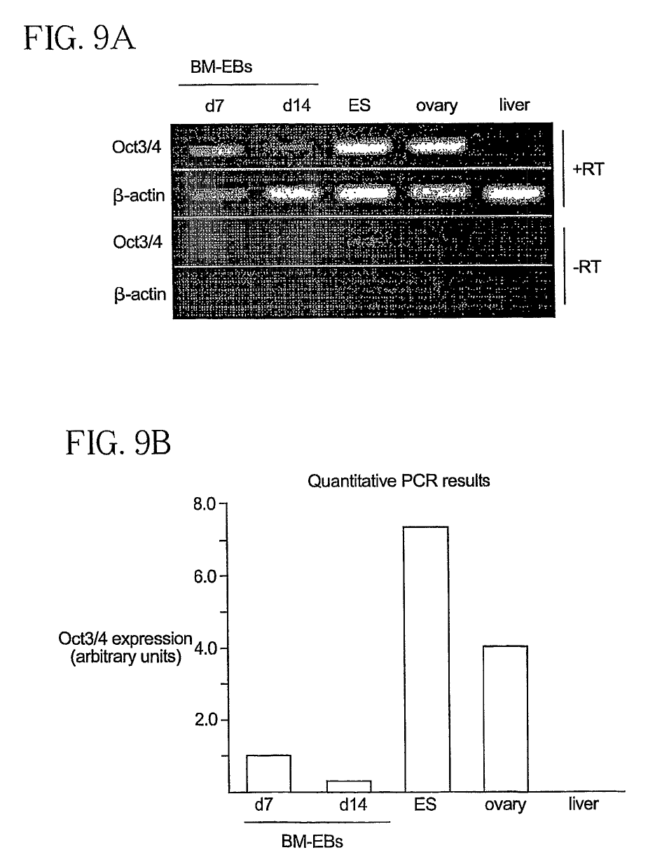

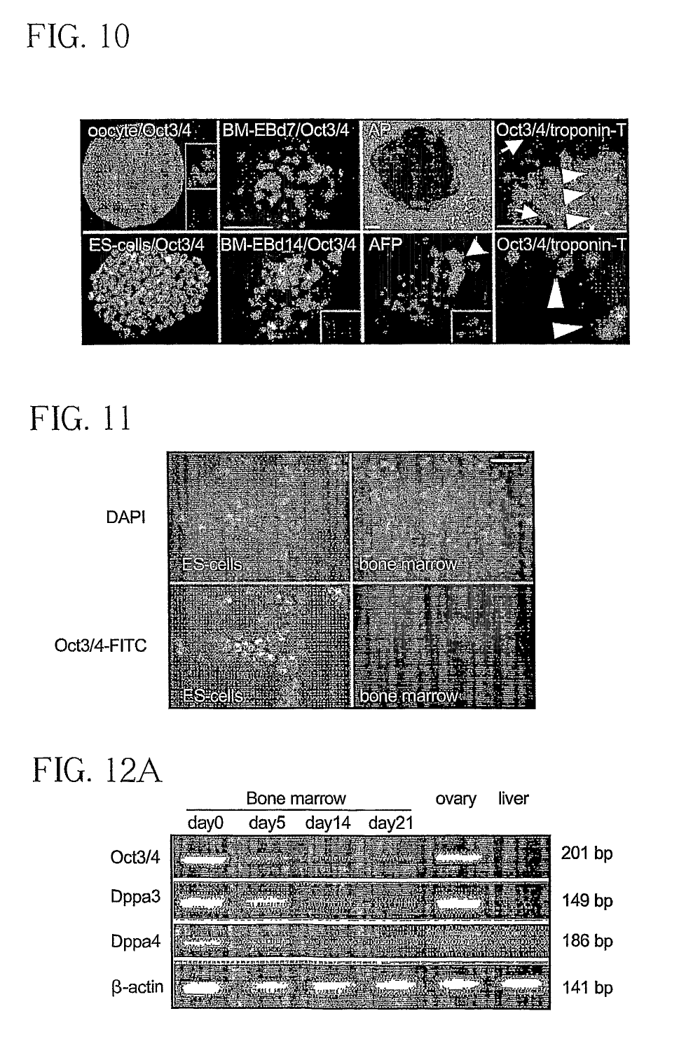

Bone marrow-derived aggregates were morphologically similar to ES-cell-derived EBs (FIGS. 8A and 8B) and expressed Oct-3/4 (FIGS. 9A and 9B), a POU-domain transcription factor that is associated with pluripotent stem cell capacity (Pesce et al., Cells Tissues Organs 165: 144-52 (1999)), and is strongly expressed in female germ cells, ES-cells and in early EBs (Rathjen et al. J. Cell. Sci. 112:601-612 (1999)); Boheler et al., Circ. Res. 91: 189-201 (2002)). Immunostaining confirmed the presence of Oct-3/4⁺ cells in the bone marrow-derived cell aggregates (FIGS. 10B, 10D, 10F and 10H), indicating that these aggregates may originate from stem cells and may function like ES-cell-derived EBs. Indeed molecular and phenotypic analysis also showed down-regulation of Oct-3/4 in bone marrow-derived aggregates during differentiation, with lower levels of expression (70% reduction; FIG. 9B) and a more patchy distribution in day-14 cell aggregates (Oct-3/4⁺ cells: 95% on day-7; but 25% on day-14; FIGS. 10B and 10F), similar to what has been previously described in ES cell-derived EBs during cardiomyogenic differentiation (Leahy et al. J. Exp. Zool. 284: 67-81 (1999)).

Double immunostaining of bone marrow-derived aggregates for Oct-3/4 and cardiac troponin-T further confirmed that bone marrow-derived cardiac myocytes originate from Oct-3/4⁺ cells present in the bone marrow-EBs (FIGS. 10D and 10H). The data obtained showed that about ⅓ of total troponin-T+cardiac myocytes found in the cell aggregates co-expressed Oct-3/4. The percent troponin-T⁺/Oct-3/4⁺ cells was 2.9±1.9%, whereas the percentage of troponin-T⁺/total Oct-3/4^{+/-} cells was 10.8±1.9%. Oct-3/4 down-regulation during cardiac myocyte generation and the proximity of troponin-T⁺/Oct-3/4⁺ cells to troponin-T⁺/Oct-3/4⁻ cells suggest that the Oct-3/4 expression state represents different stages of cardiac myocyte differentiation.

In addition to the expression of Oct-3/4 the bone marrow-derived aggregates were also positive for alkaline phosphatase (FIG. 10C), an ES-cell marker that is down-regulated during the development of cardiogenic mesoderm and ectoderm in the ES cell-derived EBs (Berstine et al., Proc. Natl. Acad. Sci. U.S.A. 70: 3899-3903 (1973)). Moreover, the bone marrow-derived EB-like aggregates also expressed alpha-fetoprotein, a primitive endoderm marker, in peripheral areas where cardiac specification occurred (FIG. 10G). This observation is consistent with observations in ES cell-derived EBs, where underlying primitive endoderm activates the differentiation of cardiomyogenic mesoderm (Bader et al., Differentiation. 68, 31-43 (2001)) prior to the appearance of spontaneous beating activity (Abe et al., Exp. Cell Res. 229, 27-34 (1996)). Indeed, the induction of both endodermal and mesodermal markers reveals that the aggregates arising from the bone marrow cells may have multi-lineage potential, a characteristic similar to ES-cell derived EBs.

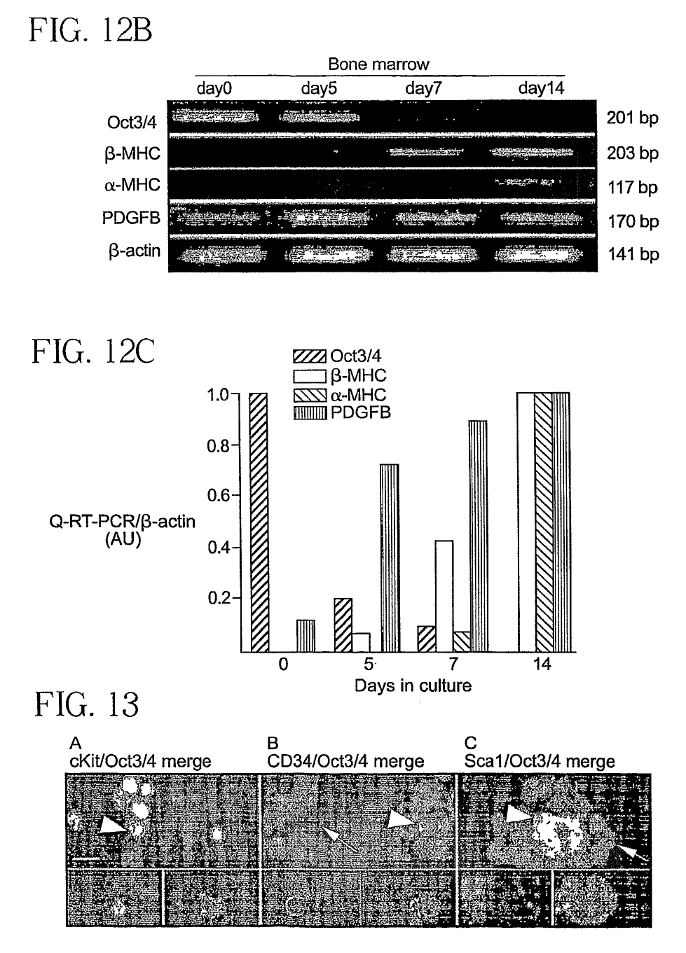

The development of bone-marrow-derived embryoid body-like cell clusters with expression of pluripotent associated markers suggests that populations of Oct3/4⁺ cells are present in adult bone marrow. In situ immunostaining of fresh isolates of bone marrow cells revealed that mouse bone marrow contains cells that express the pluripotency marker Oct-3/4, though Oct-3/4 expression is rarer (0.05±0.03%) and it is expressed at lower levels than in ES-cells (FIG. 11A-D). Further analysis of the expression of Oct-3/4, as well as the other pluripotency-associated genes, Dppa3/Stella/PGC7 and Dppa4, demonstrated a temporally inverse correlation with the induction of cardiac myocyte genes α- and β-Myosin Heavy Chain (FIGS. 12B and 12C). Similar results have been described for ES cell-derived cardiac myocytes (Bader et al. Differentiation 68: 31-43 (2001)). The expression of Oct-3/4 was also inversely correlated with platelet-derived growth factor (PDGF)-B (FIG. 12B). PDGF-B can promote cardiac myocyte differentiation from adult bone marrow cells in vitro as well as in vivo (Xaymardan et al., Circ. Res. 94: E39-E45 (2004)).

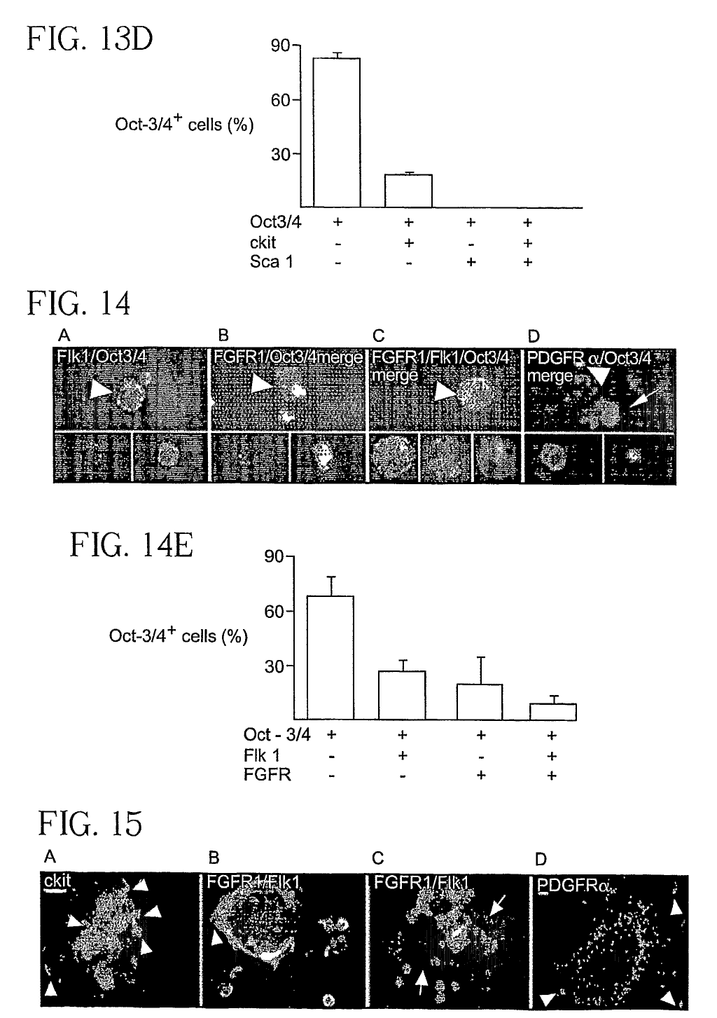

In order to identify the cells giving rise to the Oct3/4⁺ embryoid body (EB)-like structures, bone marrow cells were probed for well-characterized stem cell surface markers and receptors for cytokines VEGF and FGF-2. Immunostaining revealed that the Oct3/4⁺ bone marrow cells stained for c-Kit, but not CD34 and Sca1 (FIGS. 13A, 13B, 13C). These results are in contrast to a previous report showing expression of Sca1, but not c-Kit, in human Oct3/4⁺ bone marrow cells (D'Ippolito et al., 2004). However, the results are consistent with mouse data showing a lack of Oct3/4 expression in Sca1⁺/c-Kit bone marrow cells (Baddoo et al., 2003) and c-Kit expression in a sub-population of Oct3/4⁺ ES-cells with great developmental plasticity (Hubner et al., 2003). Bone marrow EBs were also c-Kit⁺.

Murine bone marrow Oct-3/4+ cells were also analyzed for receptors for trophic factors that support the generation of cardiac myocytes in culture (FIG. 14A-14E). Double and triple staining results showed that a proportion of Oct-3/4+ cells stained for the VEGF receptor, Flk-1 (FIGS. 14A and 14C), or/and FGF receptor (FGFR)-1 (FIGS. 14B and 14C) and were distinct from the PDGF receptor (PDGFR)α+ population of cells (FIG. 14D), suggesting that bone marrow Oct-3/4⁺ cells are a diverse population of cells where a combination of autocrine and paracrine cell communications may mediate the generation of bone marrow-derived cardiac myocytes. Indeed, cells found in the developing bone marrow-derived aggregates were also c-Kit+, FGFR-1+ and Flk-1+, showing they retained phenotypic features of the bone marrow-derived Oct-3/4+ cells they originate from (FIG. 15A-15D). The Flk1 signal was intense and widespread in day 7 to 14 bone marrow EBs and in cells surrounding clusters of beating cells. PDGFRα⁺ cells were also found adjacent to the cell aggregates, which is consistent with their role in supporting cardiac myocyte-endothelial cell communication during development (Edelberg et al., J. Clin. Invest. 102: 837-43 (1998).

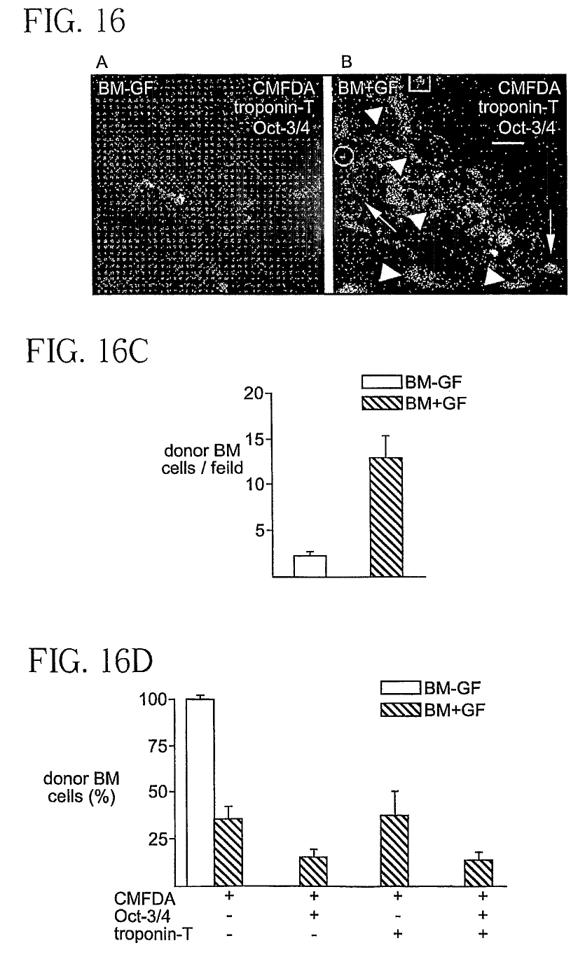

The association of growth factor receptors offers insight into the potential molecular mechanisms governing the differentiation of bone marrow-derived cardiac myocytes. Previous studies have demonstrated that FGF-2/FGFR-1 interactions are required for embryonic cardiomyogenesis (Esner et al., Int. J. Dev. Biol. 46:817-825 (2002), as compared with transdifferentiation pathways that are independent of FGF-2 activation (Condorelli et al., Proc. Natl. Acad. Sci. U.S.A. 98: 10733-38 (2001). The localization of Flk-1 in the cells found at the periphery of cardiomyogenic bone marrow-derived aggregates as well as in spontaneously beating clusters (FIG. 16B) suggests a potentially close temporal and spatial correlation between primitive endothelial cells and developing cardiomyocytes. This is consistent with the close correlation observed between angiogenesis and cardiac tissue development in the embryo (Leahy et al. J. Exp. Zool. 284:67-81 (1999), as well as endothelial progenitor cell-mediated neovascularization and infarcted heart tissue regeneration, in the adult (Xaymardan et al., J. Exp. Med. 199: 797-804 (2004)). Moreover, the association of PDGF pathways with the induction of bone marrow-derived cardiac myocytes suggests that paracrine interactions between PDGFRα+ cells and Oct3/4+ cell aggregates from the bone marrow may parallel the regulation of cardiac myocytes and endothelial cells in the developing heart.

The ability of Oct-3/4+ bone marrow cells to generate cardiac myocytes in vivo was assessed using a rat model of myocardial infarction (Xaymardan et al., J. Exp. Med. 199: 79-804 (2004)). Based on the role of anti-apoptotic pathways in the survival and function of cardiac stem cells (Bock-Marquette et al. Nature 432: 466-72 (2004)), bone marrow cells were co-delivered with an anti-apoptotic growth factor combination (GF=PDGF−AB+VEGF+Angiogenin-2) to enhance donor-derived bone marrow cell survival (FIG. 16A-D). The data obtained confirmed that trophic treatment increased donor bone marrow cell survival rates (control=no growth factors) and demonstrated that surviving bone marrow cells represent an enriched Oct-3/4+ population of cells with approximately 29.4±7.5 percent Oct-3/4+ cells, 600-fold higher than the ones found in the bone marrow. Moreover, about half (51.6±13.3%) of donor bone marrow cells (CMFDA⁺ cells) gave rise to cardiac myocytes (troponin-T⁺ cells) and approximately one-third of the de novo cardiac myocytes co-stained for Oct-3/4⁺ cells (CMFDA⁺/troponin-T+/Oct-3/4⁺ cells=14.2±4.7%). In addition, CMFDA⁻/Oct-3/4⁺/troponin-T⁺ cells were also observed (FIG. 16C) though they were fewer than CMFDA⁺/Oct-3/4⁺/troponin-T⁺ cells (0.2±0.1 versus 2.3±0.9, cells/field) suggesting that host-derived Oct-3/4+ stem cells, under these experimental conditions, may contribute to cardiac tissue regeneration, but less efficiently than injected stem cells.

Overall, the in vitro generation of the bone marrow-derived cardiac myocytes from Oct-3/4⁺ EB-like cell clusters provides important mechanistic insights into the cardiomyogenic potential of adult bone marrow cells. The generation of cardiac myocytes from the Oct-3/4⁺ cell clusters in the absence of pre-existing heart cells demonstrates that this process is not mediated by fusion nor by transdifferentiation of other mature cell types. Importantly, the in vivo experiments confirming the capacity of the bone marrow-derived Oct-3/4+ cells to give rise to cardiac myocytes in the rodent heart suggest that these cells in the endogenous bone marrow may be a source of the resident stem cells identified in cardiac myocardial tissue (Beltrami et al., Cell 114: 763-76). While the origin of the Oct-3/4+ cells remains to be defined, their presence in the bone marrow suggests that they may be preserved and/or propagated in this niche as multipotent, and possibly pluripotent, stem cells and are sustained, at least in part, in adulthood. Therefore, the present studies support the use of autologous bone marrow-cells for the cardiac tissue regeneration.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### REFERENCES

1. Makino S, Fukuda K, Miyoshi S, Konishi F, Kodama H, Pan J, Sano M, Takahashi T, Hori S, Abe H, Hata J, Umezawa A, Ogawa S. Cardiomyocytes can be generated from marrow stromal cells in vitro. J Clin Invest. 103, 697-705 (1999).
2. Jackson K A, Majka S M, Wang H, Pocius J, Hartley C J, Majesky M W, Entman M L, Michael L H, Hirschi K K, Goodell M A. Regeneration of ischemic cardiac muscle and vascular endothelium by adult stem cells. J Clin Invest. 107, 1395-1402 (2001).
3. Orlic D, Kajstura J, Chimenti S, Limana F, Jakoniuk I, Quaini F, Nadal-Ginard B, Bodine D M, Leri A, Anversa P. Mobilized bone marrow cells repair the infarcted heart, improving function and survival. Proc Natl Acad Sci USA. 98, 10344-10349 (2001).
4. Assmus B, Schachinger V, Teupe C, Britten M, Lehmann R, Dobert N, Grunwald F, Aicher A, Urbich C, Martin H, Hoelzer D, Dimmeler S, Zeiher A M. Transplantation of Progenitor Cells and Regeneration Enhancement in Acute Myocardial Infarction (TOPCARE-AMI). Circulation. 106, 3009-3017 (2002).
5. Strauer B E, Brehm M, Zeus T, Kostering M, Hernandez A, Sorg R V, Kogler G, Wernet P. Repair of Infarcted Myocardium by Autologous Intracoronary Mononuclear Bone Marrow Cell Transplantation in Humans. Circulation. 106, 1913-1918 (2002).
6. Alvarez-Dolado M, Pardal R, Garcia-Verdugo J M, Fike J R, Lee H O, Pfeffer K, Lois C, Morrison S J, Alvarez-Buylla A. Fusion of bone-marrow-derived cells with Purkinje neurons, cardiomyocytes and hepatocytes. Nature. 425, 968-973 (2003).
7. Terada N, Hamazaki T, Oka M, Hoki M, Mastalerz D M, Nakano Y, Meyer E M, Morel L, Petersen B E, Scott E W. Bone marrow cells adopt the phenotype of other cells by spontaneous cell fusion. Nature. 416, 542-545 (2002).
8. Eisenberg L M, Burns L, Eisenberg C A. Hematopoietic cells from bone marrow have the potential to differentiate into cardiomyocytes in vitro. Anat Rec. 274, 870-882 (2003).
9. Xaymardan M, Tang L, Zagreda L, Pallante B, Zheng J, Chazen J L, Chin A, Duignan I, Nahirney P, Rafii S, Mikawa T, Edelberg J M. Multimedia article. Platelet-derived growth factor-AB promotes the generation of adult bone marrow-derived cardiac myocytes. Circ Res. 94, E39-E45 (2004).
10. Cheng L, Hammond H, Ye Z, Zhan X, Dravid G. Human adult marrow cells support prolonged expansion of human embryonic stem cells in culture. Stem Cells. 21, 131-42 (2003).
11. Pesce M, Anastassiadis K, Scholer H R. Oct-4: lessons of totipotency from embryonic stem cells. Cells Tissues Organs. 165, 144-152 (1999).
12. zur Nieden N I, Ruf L J, Kempka G, Hildebrand H, Ahr H J. Molecular markers in embryonic stem cells. Toxicol In Vitro. 15, 455-461 (2001).
13. Rathjen J, Lake J A, Bettess M D, Washington J M, Chapman G, Rathjen P D. Formation of a primitive ectoderm like cell population, EPL cells, from ES cells in response to biologically derived factors. J Cell Sci. 112, 601-612 (1999).
14. Leahy A, Xiong J W, Kuhnert F, Stuhlmann H. Use of developmental marker genes to define temporal and spatial patterns of differentiation during embryoid body formation. J Exp Zool. 284, 67-81 (1999).
15. Berstine E G, Hooper M L, Grandchamp S, Ephrussi B. Alkaline phosphatase activity in mouse teratoma. Proc Natl Acad Sci USA. 70, 3899-3903 (1973).
16. Bader A, Gruss A, Hollrigl A, Al-Dubai H, Capetanaki Y, Weitzer G. Paracrine promotion of cardiomyogenesis in embryoid bodies by LIF modulated endoderm. Differentiation. 68, 31-43 (2001).
17. Abe K, Niwa H, Iwase K, Takiguchi M, Mori M, Abe S I, Abe K, Yamamura K I. Endoderm-specific gene expression in embryonic stem cells differentiated to embryoid bodies. Exp Cell Res. 229, 27-34 (1996).
18. Monzo M, de Anta J M, Peris B, Ruano D. Alpha-fetoprotein in tumours derived from cystic and simple embryoid bodies. Arch Ital Anat Embriol. 96, 113-120 (1991).
19. Bortvin A, Eggan K, Skaletsky H, Akutsu H, Berry D L, Yanagimachi R, Page D C, Jaenisch R. Incomplete reactivation of Oct4-related genes in mouse embryos cloned from somatic nuclei. Development. 130, 1673-1680 (2003).
20. D'Ippolito G, Diabira S, Howard G A, Menei P, Roos B A, Schiller P C. Marrow-isolated adult multilineage inducible (MIAMI) cells, a unique population of postnatal young and old human cells with extensive expansion and differentiation potential. J Cell Sci. 117, 2971-2981 (2004).
21. Baddoo M, Hill K, Wilkinson R, Gaupp D, Hughes C, Kopen G C, Phinney D G. Characterization of mesenchymal stem cells isolated from murine bone marrow by negative selection. J Cell Biochem. 89, 1235-1249 (2003).
22. Hubner K, Fuhrmann G, Christenson L K, Kehler J, Reinbold R, De La Fuente R, Wood J, Strauss J F 3rd, Boiani M, Scholer H R. Derivation of oocytes from mouse embryonic stem cells. Science. 300, 1251-1256 (2003).
23. Rafli S, Lyden D. Therapeutic stem and progenitor cell transplantation for organ vascularization and regeneration. Nat. Med. 9, 702-712 (2003).
24. Esner M, Pachernik J, Hampl A, Dvorak P. Targeted disruption of fibroblast growth factor receptor-1 blocks maturation of visceral endoderm and cavitation in mouse embryoid bodies. Int J Dev Biol. 46, 817-825 (2002).
25. Condorelli G, Borello U, De Angelis L, Latronico M, Sirabella D, Coletta M, Galli R, Balconi G, Follenzi A, Frati G, Cusella De Angelis M G, Gioglio L, Amuchastegui S, Adorini L, Naldini L, Vescovi A, Dejana E, Cossu G. Cardiomyocytes induce endothelial cells to trans-differentiate into cardiac muscle: implications for myocardium regeneration. Proc Natl Acad Sci USA. 98, 10733-10738 (2001).
26. D'Ippolito G, Diabira S, Howard G A, Menei P, Roos B A, Schiller P C. Marrow-isolated adult multilineage inducible (MIAMI) cells, a unique population of postnatal young and old human cells with extensive expansion and differentiation potential. J Cell Sci. 117, 2971-2981 (2004).
27. Makino S, Fukuda K, Miyoshi S, Konishi F, Kodama H, Pan J, Sano M, Takahashi T, Hori S, Abe H, Hata J, Umezawa A, Ogawa S. Cardiomyocytes can be generated from marrow stromal cells in vitro. J Clin Invest. 103, 697-705 (1999).
28. Wreggett K A, Hill F, James P S, Hutchings A, Butcher G W, Singh P B (1994). A mammalian homologue of Drosophila heterochromatin protein 1 (HP1) is a component of constitutive heterochromatin. Cytog. Cell Genet., 66: 99-103. fusion. Nature. 416, 542-545 (2002).

All publications and patents are incorporated by reference herein, as though individually incorporated by reference. The invention is not limited to the exact details shown and described, for it should be understood that many variations and modifications may be made while remaining within the spirit and scope of the invention defined by the claims.

Certain embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. An isolated mammalian bone-marrow derived pluripotent stem cell that expresses Oct3/4.

2. The cell of claim 1 that further expresses at least one of Dppa3/Stella, Dppa4, Dppa5 or a combination thereof.

3. The cell of claim 1 that further expresses c-Kit.

4. The cell of claim 1 that further expresses at least one receptor selected from the group consisting of a receptor for vascular endothelial growth factor receptor (VEGF) or fibroblast growth factor (FGF).

5. The cell of claim 4, wherein the receptor is VEGF receptor-2/fetal liver kinase 1 (Flk-1), fibroblast growth factor-1 (FGFR-1) or a combination thereof.

6. The cell of claim 1 that does not express CD34, Sca1 or a combination thereof.

7. The cell of claim 1 that has alkaline phosphatase activity.

8. The cell of claim 1 that is a Sca1⁻/CD34⁻/cKit⁺/Flk1⁺/FGFR⁺ cell.

9. The cell of claim 1 that is Oct3/4⁺ Sca1⁻CD34⁻ c-Kit^{+/-}Flk1^{+/-}FGFR^{+/-}.

10. The cell of claim 1 that is a bipotent cell.

11. The cell of claim 1 that is a cardiomyocyte progenitor cell.

12. An isolated mammalian bone-marrow derived stem cell that is Oct3/4⁺/Dppa3/Stella⁺/Dppa4⁺/Sca1⁻/CD34⁻/c-Kit⁺/Flk1⁺/FGFR⁺.

13. An isolated mammalian bone marrow derived cell that is Oct3/4⁺ Sca1⁻CD34⁻ c-Kit^{+/-}Flk1^{+/-}FGFR^{+/-}.

14. An isolated mammalian bone-marrow derived cardiomyocyte that expresses β myosin heavy chain, α myosin heavy chain, cardiac troponin T, or a combination thereof, wherein the cardiomyocyte was generated from a mammalian bone-marrow derived stem cell that expresses Oct3/4.

15. The cardiomyocyte of claim 14, that further expresses Oct3/4, Dppa 3/Stella, Dppa 4, β myosin heavy chain, α myosin heavy chain, or a combination thereof.

16. The cardiomyocyte of claim 14 having spontaneous beating and/or chronotropic activity.

17. An isolated bone-marrow derived embryoid body comprising a cardiomyocyte of claim 14.

18. An isolated bone-marrow derived embryoid body comprising the cell of any one of claims 1-13.

19. An isolated bone-marrow derived embryoid body comprising at least one cell that expresses Oct3/4, SSEA1, alpha-fetoprotein, or a combination thereof.

20. An isolated bone-marrow derived embryoid body comprising at least one cell that is Oct3/4⁺ Sca1⁻/CD34⁻/cKit⁺/Flk1⁺/FGFR⁺ or Oct3/4⁺Sca1⁻CD34⁻ c-Kit^{+/-}Flk1^{+/-}FGFR^{+/-}.

21. The bone-marrow derived embryoid body of any one of claims 18-20 that has at least one cell with alkaline phosphatase activity.

22. A composition comprising a pharmaceutically acceptable carrier and mammalian cardiomyocytes derived from Oct3/4⁺ bone-marrow cells, wherein the cardiomyocytes express at least one marker selected from the group Oct3/4, Dppa 3/Stella, Dppa 4, FGFR-1, VEGFR, flk1, c-kit, alpha fetoprotein (AFP), β myosin heavy chain and α myosin heavy chain.

23. The composition of claim 22, wherein the cardiomyocytes do not express CD34 or Sca1.

24. A method of making a cardiomyocyte comprising:
(a) obtaining a stem cell that expresses Oct3/4 from mammalian bone-marrow; and
(b) culturing the cells in a medium comprising an appropriate amount of at least one growth factor under appropriate conditions for a sufficient period of time to promote differentiation of the stem cell into a cardiomyocyte.

25. The method of claim 24, wherein the growth factor is vascular endothelial growth factor (VEGF), fibroblast growth factor-2 (FGF-2), or a combination thereof.

26. A method for making a bone-marrow derived embryoid body comprising:
(a) obtaining cardiomyocyte precursor cells from mammalian bone marrow, wherein the cells express Oct3/4; and
(b) culturing the cells in a medium comprising an appropriate amount of at least one growth factor under appropriate conditions for a sufficient period of time to provide an embryoid body.

27. The method of claim 26, wherein the bone-marrow derived embryoid body comprises at least one cell that expresses Oct3/4.

28. The method of claim 26, wherein the bone-marrow derived embryoid body further comprises at least one cell that is expresses at least one of Dppa 3/Stella or Dppa4.

29. The method of claim 26, wherein the bone-marrow derived embryoid body comprises at least one cell that expresses c-Kit.

30. The method of claim 26, wherein the bone-marrow derived embryoid body comprises at least one cell that expresses a receptor selected from the group consisting of a receptor for vascular endothelial growth factor (VEGF) or fibroblast growth factor (FGF).

31. The method of claim 26, wherein the bone-marrow derived embryoid body comprises at least one cell that expresses VEGF receptor-2/fetal liver kinase 1 (Flk-1) or fibroblast growth factor-1 (FGFR-1).

32. The method of claim 26, wherein the bone-marrow derived embryoid body comprises at least one cell having alkaline phosphatase activity.

33. A method of making an embryoid body comprising:
(a) obtaining Oct3/4⁺ cells from mammalian bone-marrow; and
(b) culturing the cells under conditions suitable to produce an embryoid body that comprises cells at least some of which are Sca1⁻CD34⁻ cKit⁺Flk1⁺FGFR⁺ or Sca1⁻CD34⁻ c-Kit^{+/-}Flk1^{+/-}FGFR^{+/-}.

34. A method for treating cardiac dysfunction in a subject having or at risk for developing cardiac dysfunction comprising administering to the subject a therapeutically effective amount of bone-marrow derived stem cells or differentiated cardiomyocytes derived from the stem cells, wherein at least one stem cell or cardiomyocyte expresses at least one marker selected from the group Oct3/4, Dppa 3/Stella, Dppa 4, FGFR-1, VEGFR, flk1, c-kit, alpha fetoprotein (AFP), β myosin heavy chain and α myosin heavy chain.

35. The method of claim 34, wherein the subject is a mammal.

36. The method of claim 34, wherein the subject is a human.

37. The method of claim 34, wherein the stem cells express Oct3/4.

38. The method of claim 34, wherein the cardiac dysfunction is myocardial infarction, ischemia, peripheral vasculature disorder (PVD), stroke, atherosclerosis, arrhythmia, heart failure, tachycardia, or congestive heart failure.

39. A method for treating a cardiac dysfunction in a mammal comprising:
(a) obtaining Oct3/4⁺ cardiomyocyte precursor cells from bone marrow stem cells collected from the mammal; and
(b) administering the cardiomyocyte precursor cells to the mammal.

40. A method for treating a cardiac dysfunction in a mammal comprising:
(a) obtaining Oct3/4⁺ cardiomyocyte precursor cells from bone marrow derived cells collected from the mammal;
(b) culturing the Oct3/4⁺ cardiomyocyte precursor cells in a culture medium comprising VEGF and FGF-2 under conditions that induce the cells to differentiate into cardiomyogenic cells;
(c) monitoring the differentiation state of the cardiomyogenic cells; and
(d) administering the cardiomyogenic cells to the mammal.

41. An gamete-like cell generated in vitro from bone-marrow derived Oct3/4⁺ stem cells.

42. The gamete-like cell of claim 41 that is Sca1⁻ CD34⁻ cKit⁺ Flk1⁺ FGFR⁺ or Sca1⁻ CD34⁻ c-Kit^{+/-} Flk1^{+/-} FGFR^{+/-}.

43. An oocyte-like cell generated in vitro from bone-marrow derived Oct3/4⁺ stem cells.

44. The oocyte-like cell of claim 43 that is Sca1⁻ CD34⁻ cKit⁺ Flk1⁺ FGFR⁺ or Sca1⁻ CD34⁻ c-Kit^{+/-} Flk1^{+/-} FGFR^{+/-}.
